(19) United States
(12) Patent Application Publication
(10) Pub. No.: US 2014/0018252 A1
(43) Pub. Date: Jan. 16, 2014

(54) GENE ARRAY TECHNIQUE FOR PREDICTING RESPONSE IN INFLAMMATORY BOWEL DISEASES

(71) Applicant: Children's Hospital Medical Center, Cincinnati, OH (US)

(72) Inventors: Huan Xu, Cincinnati, OH (US); Lee A. Denson, Wyoming, OH (US); Bruce Aronow, Cincinnati, OH (US)

(73) Assignee: Children's Hospital Medical Center, Cincinnati, OH (US)

(21) Appl. No.: 13/857,351

(22) Filed: Apr. 5, 2013

Related U.S. Application Data
(63) Continuation of application No. 12/445,764, filed on Jun. 16, 2010, now abandoned.
(60) Provisional application No. 60/852,364, filed on Oct. 17, 2006.

(51) Int. Cl. C12Q 1/68

(52) U.S. Cl. CPC C12Q 1/6883
USPC 506/9; 435/6.12; 506/16

(57) ABSTRACT

Disclosed are methods for classifying individuals having or suspected of having an inflammatory bowel disease, such as Crohn's Disease or Ulcerative Colitis, as ‘responders’ or ‘non-responders’ to first-line treatment, generally comprising the steps of a) obtaining a biological sample from the individual, b) isolating mRNA from the biological sample c) determining a gene expression profile from the biological sample; and d) comparing the gene expression profile of the individual to a reference gene expression profile or other suitable control such that changes in expression can be used to stratify individuals and predict efficacy of first-line therapy. A gene expression system is further provided for carrying out these methods.

### BACKGROUND OF THE INVENTION

Inflammatory Bowel Disease or “IBD” is a collective term used to describe diseases including Crohn's disease (CD), ulcerative colitis (UC), microscopic colitis, and indeterminate colitis. Most IBD can be categorized as either CD or UC. With current diagnostic approaches, approximately 60% of IBD patients are classified as CD, 30% as UC, and 10% as indeterminate colitis (IC). The occurrence of IBD is estimated to be as high as up to approximately 2,000,000 Americans, at a cost of greater than $2 billion dollars annually.

CD is characterized by discontinuous transmural inflammation that can involve any part of the gastrointestinal (GI) tract, although the terminal ileum and proximal colon are most commonly involved. This inflammation can result in strictures, microperf orations, and fistulae. The inflammation is noncontiguous and thus can produce skip lesions throughout the bowel. Histologically, CD can have either transmural lymphoid aggregates or non-necrotizing granulomas. Although granulomas are pathognomonic, they are seen in only 40% of patients with CD. In contrast, UC is characterized by continuous superficial inflammation limited to the colon, beginning in the rectum and extending proximally.

Both CD and UC are chronic and most frequently have their onset in early adolescence or early adult life. The cause of IBD is unclear, though it is speculated that both environmental and genetic factors play a role. See Collins, P. et al, Ulcerative colitis: Diagnosis and Management” BMJ Vol. 333, 12 Aug. 2006 and Hanauer, S. Inflammatory Bowel Disease: Epidemiology, pathogenesis, and Therapeutic Opportunities. Inflamm. Bowel Dis. 2006 January; 12 Suppl. 1:53-9. Review. The most common symptom of both UC and CD is diarrhea, sometimes accompanied by abdominal cramps, tenesmus (straining at stool), blood, fever, fatigue, and loss of appetite. Some patients have alternating periods of remission with relapse or flare. Other patients have continuous symptoms without remission due to continued inflammation. The severity and responsiveness to treatment for IBD varies widely from individual to individual.

### Diagnosis

The diagnosis of UC or CD is established by finding characteristic intestinal ulcerations and excluding alternative diagnoses, such as enteric infections or ischemia. Active disease in UC is characterized by the endoscopic appearance of superficial ulcerations, friability, a distorted mucosal vascular pattern, and exudate. Patients with severely active disease can have deep ulcers and friability that result in spontaneous bleeding. The typical distribution of disease is continuous from the rectum proximally. However, patients with partially treated UC may have discontinuous or patchy involvement.

The ulcerations of CD may appear aphthoid, but could also be deep and serpiginous. Skip areas, a “cobblestone” appearance, pseudopolyps, and rectal sparing are characteristic findings. Air contrast barium enema, small-bowel series, or colonoscopy may demonstrate these typical lesions. On a small-bowel series, CD often is manifested by separation of bowel loops and a narrowed-terminal ileal lumen, the so-called “string sign.”

Histologic features of UC include disease limited to the mucosa and submucosa, mucin depletion, ulcerations, exudate, and crypt abscesses. In CD, non-necrotizing granulomas, transmural lymphoid aggregates, and microscopic skip lesions can be seen. Typical lesions of CD also may be seen in the upper gastrointestinal tract. The inflammation is localized in the ileocecal region in 50% of cases, the small bowel in 25% of cases, the colon in 20% of cases, and the upper gastrointestinal tract or perirectum in 5%.

### Assessment of Disease Activity

Disease activity including response to treatment or remission of disease in patients having UC may be assessed using the Clinical Activity Disease Index developed in 1955 by Truelove and Witts (See “Cortisone in ulcerative colitis: final report on a therapeutic trial,” BMJ 1955; 2: 1041-1048; See also Table 1). Patients with fulminant or toxic colitis usually have more than 10 bowel movements per day, continuous bleeding, abdominal distention and tenderness, and radiologic evidence of edema and possibly bowel dilation.

TABLE 1
Trulove and Witts Criteria for Assessing Disease Activity in Ulcerative
Colitis

| Criteria | Mild Activity | Severe Activity |
|---|---|---|
| Daily bowel movements (no.) | < or = to 5 | >5 |
| Hematochezia | Small amounts | Large amounts |
| Temperature | <37.5° C. | > or = to 37.5° C. |
| Pulse | <90/min | > or = 90/min |
| Erythrocyte sedimentation rate | <30 mm/h | > or = to 30 mm/h |
| Hemoglobin | >10 g/dl | < or = to 10 g/dl |

Patients with fewer than all 6 of the above criteria for severe activity have moderately active disease.

The severity of disease in CD patients may be determined using several clinical disease activity indices. For example, the Crohn's Disease Activity Index (CDAI) developed by Best et al. is often used in clinical trials to measure disease activity. (See Best W R, Becktel—A—J M, Singleton J W. “Rederived values of the eight coefficients of the Crohn's Disease Activity Index (CDAI),” Gastroenterology. 1979;77:843-846; Hyams J S, et al., “Development and Validation of a Pediatric Crohn's Disease Activity Index” J. Pediatric Gastroenterol. Nutr. 1991; 12:439-47; Hanauer S P et al, “Maintenance infliximab for Crohn's disease, the ACCENT I Randomized Trial” Lancet 2002; 359:1541-9, both incorporated herein by reference.) The index consists of eight factors, each summed after adjustment with a weighting factor. The components of the CDAI and weighting factors are listed in Table 2:

TABLE 2

| | Weighting |
|---|---|
| Clinical or laboratory variable | factor |
| Number of liquid or soft stools | x 2 |
| each day for seven days | |
| Abdominal pain (graded from 0-3 on severity) | x 6 |
| General well being, subjectively assessed | x 6 |
| from 0 (well) to 4 (terrible) | |
| Presence of complications* | x 30 |
| Number of infirm days | x 5 |
| (interpreted as non-functional days) | |
| Presence of an abdominal mass | x5 |
| (0 as none, 2 as questionable, 5 as definite) | |
| Hematocrit of <0.47 in men | x 6 |
| and <0.42 in women | |
| Percentage deviation from standard weight | x 1 |

*The complications were listed as follows: the presence of joint pains (arthralgia) or frank arthritis; inflammation of the iris (uveitis); the presence of erythema nodosum or pyoderma gangrenosum; aphthous ulcers; anal fissures, fistulae or abscesses; or fever over the previous week.

Remission of CD is defined as an absolute value of the CDAI of less than 150, while severe disease is defined as a value of greater than 450 in adults. Most major research studies on medications in CD define response as a fall of the CDAI of greater than 70 points. In pediatric patients, disease activity is measured in clinical trials using the PCDAI, and remission is defined as an absolute value of 10 or less, with moderate disease defined as greater than or equal to 30. Response in pediatric patients is defined as a fall of the PCDAI of 12.5 points.

Alternatively, the Harvey-Bradshaw index may be used to assess disease activity. The Harvey-Bradshaw index was devised in 1980 as a simpler version of the CDAI for data collection purposes. The index is described in Harvey R, Bradshaw J (1980). “A simple index of Crohn's-disease activity.” Lancet 1 (8167): 514, incorporated herein by reference. It consists of only clinical parameters listed in Table 3.

TABLE 3
Harvey-Bradshaw Index Clinical Parameters

general well-being (0 = very well, 1 = slightly below average,
2 = poor, 3 = very poor, 4 = terrible)
abdominal pain (0 = none, 1 = mild, 2 = moderate, 3 = severe)
number of liquid stools per day
abdominal mass (0 = none, 1 = dubious, 2 = definite,
3 = tender)
complications, as above, with one point for each.

In addition, the PCDAI index is well-established for defining remission and mild, moderate and severely active disease in pediatric disease, as described by Hyams J S, et al., “Development and Validation of a Pediatric Crohn's Disease Activity Index” J. Pediatric Gastroenterol. Nutr. 1991; 12:439-47, incorporated herein by reference.

### Therapeutic Treatment of IBD

The current approach to the treatment of CD is sequential: first to treat acute disease, then to maintain remission. The initial treatment is directed towards treatment of infection and reduction of inflammation. Current options for induction of remission in IBD include 5-aminosalicylic acid (5-ASA) drugs, corticosteroids, methotrexate, and infliximab. Options for maintenance of remission include mesalamine, the immunomodulators 6-mercaptopurine/azathioprine (6-MP/AZA), methotrexate and infliximab. Once remission is induced, the goal of treatment becomes maintenance of remission, avoiding the return of active disease, or “flares.” Where drug therapy fails, surgery may be required.

The most common first line regiment includes induction of remission with prednisone, and maintenance of remission with 6-MP/AZA or 5-ASA. However, this treatment yields a steroid-free remission rate of only fifty percent at one year, and a significant portion of patients fail to respond to first line therapy. To date, there are currently no established clinical tests for predicting response to first line therapy, and newly diagnosed patients must first be subjected to first line therapy, despite only a 50% chance of a successful outcome. In the absence of a reliable test to predict response to therapy, patients are empirically offered agents for induction and maintenance of remission largely based upon disease severity and location. As the effectiveness of any one agent is typically on the order of 50% to 80%, this leads to a substantial number of patients receiving a series of ineffective agents, with attendant side effects, before an effective regimen is identified.

The two most widely used drug families for IBD are steroids and 5-aminosalicylic acid (5-ASA) drugs, both of which reduce inflammation of the affected parts of the intestines. A non-limiting review of therapeutics commonly used for the treatment of IBD follows below.

### Steroids

Corticosteroids are used primarily for treatment of moderate to severe flares of CD. The most commonly prescribed oral steroid is prednisone, which is typically dosed at 1.0 mg/kg for induction of remission. Intravenous steroids are used for cases refractory to oral steroids, or where the patient cannot take oral steroids. Budesonide (formulated as Entocort) is an oral corticosteroid with fewer systemic adverse effects due to 90% first-pass metabolism by the liver. Budesonide is effective as a conventional corticosteroid treatment for distal ileal and right colonic disease, but is less potent in transverse and distal colonic disease. Budesonide is also useful when used in combination with antibiotics for active CD.

### Aminosalicylates

5-aminosalicylic acid (5-ASA) drugs are also effective in inducing and maintaining remission for patients with UC, and may have a modest effect in some patients with CD. The 5-ASAs include mesalazine or mesalamine, which is marketed in the forms Asacol, Pentasa, Salofalk, Dipentum and Rowasa and, sulfasalazine (Azulfidine, Azulfidine EN-Tabs; Salazopyrin EN-Tabs, SAS in Canada; salazosulfapyridine, salicylazosulpapyridine), which is converted to 5-ASA and sulfapyridine by intestinal bacteria. The sulfapyridine may also have some therapeutic effect in addition to the 5-ASA. Two other aminosalicylates, olsalazine sodium (Dipentum) consisting of two 5-ASA moieties connected by an azobond, and balsalazide disodium (Colazal), a 5-ASA moiety attached to an inert molecule by an azobond, may be used to treat CD or UC.

### Immunosuppressive Medications

Immunosuppressive medications may also be used to treat patients with moderate to severe IBD. These include, for example, azathioprine and its active metabolite 6-mercaptopurine. Immunosuppressive drugs such as 6-mercaptopurine may be used for long-term treatment of IBD, and are particularly used for patients dependent on chronic high-dose steroid therapy. Azathioprine is a prodrug for 6-mercaptopurine, which is converted into 6-methylmercaptopurine by the enzyme thiopurine methyltransferase (TPMT) or 6-thioguanine by the enzyme hypoxanthine phosphoribosyltransferase.

Methotrexate is another immunosuppressive medication effective for induction and maintenance of remission in CD. Alternatively, cyclosporine may be used in patients with severe UC. Approximately 50% to 80% of patients refractory to intravenous corticosteroid treatment may avoid surgical treatment such as colectomy with intravenous cyclosporine treatment. Tacrolimus and mycophenolate mofetil may also be used as second-line immunosuppressive options.

TNF-Alpha Antagonists

Remicade is the first of a new class of agents for the treatment of Crohn's disease that block activity of a key biologic response mediator called tumour necrosis factor alpha (TNF-alpha). Overproduction of TNF-alpha leads to inflammation in autoimmune conditions such as Crohn's disease. It is believed that Remicade reduces intestinal inflammation in patients with Crohn's disease by binding to and neutralising TNF-alpha on the cell membrane and in the blood. Remicade is indicated for treatment of severe, active Crohn's disease in patients who have not responded despite a full and adequate course of therapy with a corticosteroid and/or an immunosuppressant, and as a treatment of fistulizing Crohn's disease in patients who have not responded despite a full and adequate course of therapy with conventional treatment.

Due to the side effects of first line therapy, the cost of treatment, and the delay in improving the quality of living among those suffering from IBD, there is an urgent and unmet need for determining the most effective course of treatment for IBD patients.

### Brief Summary

The instant disclosure generally relates to a method for classifying an individual having or suspected of having an inflammatory bowel disease as a responder or a non-responder to first-line therapy for the inflammatory bowel disease, wherein the first line therapy is one of 5-aminosalicylic acid (5-ASA) drugs, corticosteroids, methotrexate, or infliximab. The method generally comprises the steps of identifying an individual having or suspected of having an inflammatory bowel disease, such as Crohn's disease, obtaining a biological sample from the individual, isolating mRNA from the biological sample, determining the mRNA levels of one or more genes identified in any of Tables 4-8 to obtain a gene expression profile and comparing the gene expression profile to a suitable control such that the individual may be classified as a responder or a non-responder to first-line therapy. The control may be, for example, the gene expression profile of sample obtained from known responders or non-responders.

In one embodiment, gene expression is determined by PCR. In yet another embodiment, gene expression is determined by a technique using hybridization, for example, to a oligonucleotide of a predetermined sequence comprising DNA, RNA, cDNA, PNA, genomic DNA, or synthetic oligonucleotides.

In yet another embodiment, gene expression may be obtained by detection and/or measurement of the gene product, where the gene product is known or determined to reasonably correlate with gene expression.

The instant disclosure further relates to a gene expression system for identifying responders and non-responders to first line treatment for an inflammatory bowel disease in individuals having or suspected of having the disease, comprising a solid support having one or more oligonucleotides affixed to said solid support wherein the one or more nucleotides further comprises at least one sequence selected from those listed in Table 4, 5, 6, 7, or 8. The gene expression system may further comprise one or more normalization sequences and/or a reference standard. In one embodiment, the solid support comprises an array selected from the group consisting of a chip array, a plate array, a bead array, a pin array, a membrane array, a solid surface array, a liquid array, an oligonucleotide array, a polynucleotide array, a cDNA array, a microfilter plate, a membrane or a chip.

### DETAILED DESCRIPTION

### Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provide one skilled in the art with a general guide to many of the terms used in the present application.

For purposes of the present invention, the following terms are defined below.

The term “array” or “microarray” in general refers to an ordered arrangement of hybridizable array elements such as polynucleotide probes on a substrate. An “array” is typically a spatially or logically organized collection, e.g., of oligonucleotide sequences or nucleotide sequence products such as RNA or proteins encoded by an oligonucleotide sequence. In some embodiments, an array includes antibodies or other binding reagents specific for products of a candidate library. The array element may be an oligonucleotide, DNA fragment, polynucleotide, or the like, as defined below. The array element may include any element immobilized on a solid support that is capable of binding with specificity to a target sequence such that gene expression may be determined, either qualitatively or quantitatively. When referring to a pattern of expression, a “qualitative” difference in gene expression refers to a difference that is not assigned a relative value. That is, such a difference is designated by an “all or nothing” valuation. Such an all or nothing variation can be, for example, expression above or below a threshold of detection (an on/off pattern of expression). Alternatively, a qualitative difference can refer to expression of different types of expression products, e.g., different alleles (e.g., a mutant or polymorphic allele), variants (including sequence variants as well as post-translationally modified variants), etc. In contrast, a “quantitative” difference, when referring to a pattern of gene expression, refers to a difference in expression that can be assigned a value on a graduated scale, (e.g., a 0-5 or 1-10 scale, a ++++ scale, a grade 1 grade 5 scale, or the like; it will be understood that the numbers selected for illustration are entirely arbitrary and in no-way are meant to be interpreted to limit the invention). Microarrays are useful in carrying out the methods disclosed herein because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

A “DNA fragment” includes polynucleotides and/or oligonucleotides and refers to a plurality of joined nucleotide units formed from naturally-occurring bases and cyclofuranosyl groups joined by native phosphodiester bonds. This term effectively refers to naturally-occurring species or synthetic species formed from naturally-occurring subunits. “DNA fragment” also refers to purine and pyrimidine groups and moieties which function similarly but which have non naturally-occurring portions. Thus, DNA fragments may have altered sugar moieties or inter-sugar linkages. Exemplary among these are the phosphorothioate and other sulfur containing species. They may also contain altered base units or other modifications, provided that biological activity is retained. DNA fragments may also include species that include at least some modified base forms. Thus, purines and pyrimidines other than those normally found in nature may be so employed. Similarly, modifications on the cyclofuranose portions of the nucleotide subunits may also occur as long as biological function is not eliminated by such modifications.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleo tides, single- or double-stranded ribonucleotides, RNA:DNA hybrids and double-stranded DNAs. Oligonucleotides, such as single-stranded DNA oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a subject, relative to its expression in a normal or control subject. A differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes, or a comparison of the ratios of the expression between two or more genes, or even a comparison of two differently processed products of the same gene, which differ between normal subjects and subjects suffering from a disease, or between various stages of the same disease. Differential expression includes both quantitative, as well as qualitative, differences in the temporal or cellular expression pattern in a gene or its expression products. As used herein, “differential gene expression” can be present when there is, for example, at least an about a one to about two-fold, or about two to about four-fold, or about four to about six-fold, or about six to about eight-fold, or about eight to about ten-fold, or greater than about 11 told difference between the expression of a given gene in a patient of interest compared to a suitable control. However, a fold change less than one is not intended to be excluded, and to the extent such change can be accurately measured, a fold change less than one may be reasonably relied upon in carrying out the methods disclosed herein. In some embodiments, the fold change may be greater than about five or about 10 or about 20 or about 30 or about 40.

The phrase “gene expression profile” as used herein, is intended to encompass the general usage of the term as used in the art, and generally means the collective data representing gene expression with respect to a selected group of two or more genes, wherein the gene expression may be upregulated, downregulated, or unchanged as compared to a reference standard, A gene expression profile is obtained via measurement of the expression level of many individual genes. The expression profiles can be prepared using different methods. Suitable methods for preparing a gene expression profile include, but are not limited to quantitative RT-PCR, Northern Blot, in situ hybridization, slot-blotting, nuclease protection assay, nucleic acid arrays, and immunoassays. The gene expression profile may also be determined indirectly via measurement of one or more gene products (whether a full or partial gene product) for a given gene sequence, where that gene product is known or determined to correlate with gene expression.

The phrase “gene product” is intended to have the meaning as generally understood in the art and is intended to generally encompass the product(s) of RNA translation resulting in a protein and/or a protein fragment. The gene products of the genes identified herein may also be used for the purposes of diagnosis or treatment in accordance with the methods described herein.

A “reference gene expression profile” as used herein, is intended to indicate the gene expression profile, as defined above, for a preselected group which is useful for comparison to the gene expression profile of a subject of interest. For example, the reference gene expression profile may be the gene expression profile of a single individual known to not have an inflammatory bowel disease (i.e. a “normal” subject) or the gene expression profile represented by a collection of RNA samples from “normal” individuals that has been processed as a single sample. The “reference gene expression profile” may vary, and such variance will be readily appreciated by one of ordinary skill in the art.

The phrase “reference standard” as used herein may refer to the phrase “reference gene expression profile” or may more broadly encompass any suitable reference standard which may be used as a basis of comparison with respect to the measured variable. For example, a reference standard may be an internal control, the gene expression or a gene product of a “healthy” or “normal” subject, a housekeeping gene, or any unregulated gene or gene product. The phrase is intended to be generally non-limiting in that the choice of a reference standard is well within the level of skill in the art and is understood to vary based on the assay conditions and reagents available to one using the methods disclosed herein.

“Gene expression profiling” as used herein, refers to any method that can analyze the expression of selected genes in selected samples.

The phrase “gene expression system” as used herein, refers to any system, device or means to detect gene expression and includes diagnostic agents, candidate libraries, oligonucleotide sets or probe sets.

The terms “diagnostic oligonucleotide” or “diagnostic oligonucleotide set” generally refers to an oligonucleotide or to a set of two or more oligonucleotides that, when evaluated for differential expression their corresponding diagnostic genes, collectively yields predictive data. Such predictive data typically relates to diagnosis, prognosis, selection of therapeutic agents, monitoring of therapeutic outcomes, and the like. In general, the components of a diagnostic oligonucleotide or a diagnostic oligonucleotide set are distinguished from oligonucleotide sequences that are evaluated by analysis of the DNA to directly determine the genotype of an individual as it correlates with a specified trait or phenotype, such as a disease, in that it is the pattern of expression of the components of the diagnostic oligonucleotide set, rather than mutation or polymorphism of the DNA sequence that provides predictive value. It will be understood that a particular component (or member) of a diagnostic oligonucleotide set can, in some cases, also present one or more mutations, or polymorphisms that are amenable to direct genotyping by any of a variety of well known analysis methods, e.g., Southern blotting, RFLP, AFLP, SSCP, SNP, and the like.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

A “gene expression system” refers to any system, device or means to detect gene expression and includes diagnostic agents, candidate libraries oligonucleotide, diagnostic gene sets, oligonucleotide sets, array sets, or probe sets.

As used herein, a “probe” refers to the gene sequence arrayed on a substrate.

The terms “splicing” and “RNA splicing” are used interchangeably and refer to RNA processing that removes introns and joins exons to produce mature mRNA with continuous coding sequence that moves into the cytoplasm of an eukaryotic cell.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

As used herein, a “target” refers to the sequence derived from a biological sample that is labeled and suitable for hybridization to a probe affixed on a substrate.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The practice of the present invention will employ, unless otherwise indicated, conventional techniques of molecular biology (including recombinant techniques), microbiology, cell biology and biochemistry, which are within the skill of the art.

### Gene Expression Profiling

The present invention relates to a method of predicting the optimal course of therapy for patients having an inflammatory bowel disease (IBD), for example, Crohn's disease (CD) or ulcerative colitis (UC) using a diagnostic oligonucleotide set or gene expression profile as described herein, via classification of an individual having or suspected of having a inflammatory bowel disease as being either a “responder” or “non-responder” to first-line therapy. In one embodiment, the methods described herein may be used to predict the optimal course of therapy, or identify the efficacy of a given treatment in an individual having, or suspected of having an inflammatory bowel disease. In other embodiments, the methods described herein may be used to predict the optimal course of therapy post-diagnosis, for example, after treatment of an individual having an IBD has begun, such that the therapy may be changed or adjusted, in accordance with the outcome of the diagnostic methods.

The present invention also relates to diagnostic oligonucleotides and diagnostic oligonucleotide sets and methods of using the diagnostic oligonucleotides and oligonucleotide sets to diagnose or monitor disease, assess severity of disease, predict future occurrence of disease, predict future complications of disease, determine disease prognosis, evaluate the patient's risk, “stratify” or classify a group of patients, assess response to current drug therapy, assess response to current non-pharmacological therapy, identify novel therapeutic compounds, determine the most appropriate medication or treatment for the patient, predict whether a patient is likely to respond to a particular drug, and determine most appropriate additional diagnostic testing for the patient, as well as other clinically and epidemiologically relevant applications. As set forth above, the term “diagnostic oligonucleotide set” generally refers to a set of two or more oligonucleotides that, when evaluated for differential expression of their products, collectively yields predictive data. Such predictive data typically relates to diagnosis, prognosis, monitoring of therapeutic outcomes, and the like. In general, the components of a diagnostic oligonucleotide set are distinguished from nucleotide sequences that are evaluated by analysis of the DNA to directly determine the genotype of an individual as it correlates with a specified trait or phenotype, such as a disease, in that it is the pattern of expression of the components of the diagnostic nucleotide set, rather than mutation or polymorphism of the DNA sequence that provides predictive value. It will be understood that a particular component (or member) of a diagnostic nucleotide set can, in some cases, also present one or more mutations, or polymorphisms that are amenable to direct genotyping by any of a variety of well known analysis methods, e.g., Southern blotting, RFLP, AFLP, SSCP, SNP, and the like.

In another embodiment of the present invention, a gene expression system useful for carrying out the described methods is also provided. This gene expression system can be conveniently used for determining a diagnosis, prognosis, or selecting a treatment for patients having or suspected of having an IBD such as CD or UC.

In one embodiment, the methods disclosed herein allow one to classify an individual of interest as either a “responder” or a “non-responder” to first-line treatment using a gene expression profile. For purposes of the methods disclosed herein, the term “responder” refers to a patient that responds to first line therapy and does not require a second induction of remission during the year following the induction of remission. In contrast, the term “non-responder” refers to a patient having an IBD such as CD that will require a second induction of remission using any therapy. For example, treatment non-responders may require more than one course of corticosteroids, or anti-TNF, during the first year.

Thus, in accordance with the methods, a classification of an individual as a “responder” indicates that first line treatment is likely to be successful in treating the IBD, and as such, may be the treatment of choice, while an individual identified as being a non-responder would generally not be an ideal candidate for traditional first-line therapies. Rather, an individual identified as a non-responder would likely benefit from more aggressive, or second-line therapies typically reserved for individuals that have not responded to first-line treatment.

Classifying patients as either a “responder” or a “non-responder” is advantageous, in that it allows one to predict the optimal course of therapy for the patient. This classification may be useful at the outset of therapy (at the time of diagnosis) or later, when first-line therapy has already been initiated, such that treatment may be altered to the benefit of the patient.

In general, the method of using a gene expression profile or gene expression system for diagnosing an individual as a responder or a non-responder comprises measuring the gene expression of a gene identified in any of Tables 4-8 or the sequence listing. Gene expression, as used herein, may be determined using any method known in the art reasonably calculated to determine whether the expression of a gene is upregulated, down-regulated, or unchanged, and may include measurement of RNA or the gene product itself.

In one embodiment, an individual is characterized as a responder or nonresponder to first line therapy via measurement of the expression of one or more genes of Table 4 in the individual as compared to the expression of one or more genes of Table 4 in a suitable control (such as an individual previously determined to be a responder or nonresponder). In another embodiment the one or more genes are selected from Table 5. In another embodiment the one or more genes are selected from Table 6. In another embodiment the one or more genes are selected from Table 7. In another embodiment the one or more genes are selected from Table 8. The genes selected for measurement of expression may be selected on the basis of fold difference. For example, the genes may be those having a fold-change of greater than about 2 or about 3, or about 4 or about 5 as identified in any of Tables 4, 5, 6, 7, or 8.

In yet another embodiment, the method of identifying an individual having or suspected of having an inflammatory bowel disease such as comprises the steps of: 1) providing an array set immobilized on a substrate, wherein the array set comprises one or more oligonucleotides derived from the sequences listed in Tables 4-8, or the Sequence Listing, 2) providing a labeled target obtained from mRNA isolated from a biological sample from a patient having an IBD such as CD or UC, 3) hybridizing the labeled target to the array set under suitable hybridization conditions such that the labeled target hybridizes to the array elements, 4) determining the relative amounts of gene expression in the patient's biological sample as compared to a reference sample by detecting labeled target that is hybridized to the array set; 5) using the gene expression profile to classify the patient as a responder or a non-responder; and 6) predicting the optimal course of therapy based on said classification.

The one or more sequences that comprise the array elements may be selected from any of the sequences listed in Tables 4-8 or the Sequence Listing. In one embodiment, the gene expression system comprises one or more array elements wherein the one or more array elements correspond to sequences selected from those sequences listed in Tables 4-8, or the Sequence Listing. In one embodiment, the array set comprises the sequences listed in Table 5. In another embodiment, the array set comprises the sequences listed in Table 6.

The present invention also relates to an apparatus for predicting the optimal course of therapy in a patient having an inflammatory bowel disease such as CD or UC. The apparatus comprises a solid support having an array set immobilized thereon, wherein labeled target derived from mRNA from a patient of interest is hybridized to the one or more sequences of the array set on the solid support, such that a change in gene expression for each sequence compared to a reference sample or other suitable control may be determined, permitting a determination of the optimal course of therapy for the patient. The array set comprises one or more sequences selected from those listed in Tables 4-8 or the Sequence Listing described herein. In one embodiment, the array set comprises the sequences listed in Table 5. In another embodiment, the array set comprises the sequences listed in Table 6.

In yet another embodiment, the method of classifying an individual having or suspected of having an inflammatory bowel disease as a responder or non-responder comprises the steps of: 1) obtaining mRNA isolated from a biological sample from a patient having or suspected of having an inflammatory bowel disease, 2) reverse transcribing mRNA to obtain the corresponding DNA; 3) selecting suitable oligonucleotide primers corresponding to one or more genes selected from Tables 4-8 or the Sequence Listing, 4) combining the DNA and oligonucleotide primers in a suitable hybridization solution; 5) incubating the solution under conditions that permit amplification of the sequences corresponding to the primers; and 6) determining the relative amounts of gene expression in the patient's biological sample as compared to a reference sample or other suitable control; wherein the resulting gene expression profile can be used to classify the patient as a responder or a non-responder.

In other embodiments, real time PCR methods or any other method useful in measuring mRNA levels as known in the art may also be used. Alternatively, measurement of one or more gene products using any standard method of measuring protein (such as radioimmunoassay methods or Western blot analysis) may be used to determine a gene expression profile.

The methods of gene expression profiling that may be used with the methods and apparatus described herein are well-known in the art. In general, methods of gene expression profiling can be divided into methods based on hybridization analysis of polynucleotides, and methods based on sequencing of polynucleotides. Commonly used methods known in the art for the quantification of mRNA expression in a sample include northern blotting and in situ hybridization (Parker & Barnes, Methods in Molecular Biology 106:247 283 (1999)), RNAse protection assays (Hod, Biotechniques 13:852 854 (1992)), and reverse transcription polymerase chain reaction (RT-PCR) (Weis et al., Trends in Genetics 8:263 264 (1992)), or modified RT-PCR methods, such as that described in U.S. Pat. No. 6,618,679. Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In one embodiment described herein, gene array technology such as microarray technology is used to profile gene expression.

### Arrays and Microarray Technologies

Array and microarray techniques known in the art to determine gene expression may be employed with the invention described herein. Where used herein, array refers to either an array or microarray. An array is commonly a solid-state grid containing sequences of polynucleotides or oligonucleotides (array elements) of known sequences are immobilized at a particular position (also referred to as an “address”) on the grid. Microarrays are a type of array termed as such due to the small size of the grid and the small amounts of nucleotide (such as nanogram, nanomolar or nanoliter quantities) that are usually present at each address. The immobilized array elements (collectively, the “array set”) serve as hybridization probes for cDNA or cRNA derived from messenger RNA (mRNA) isolated from a biological sample. An array set is defined herein as one or more DNA fragments or oligonucleotides, as defined above, that are immobilized on a solid support to form an array.

In one embodiment, for example, the array is a “chip” composed, e.g., of one of the above specified materials. Polynucleotide probes, e.g., RNA or DNA, such as cDNA, synthetic oligonucleotides, and the like, or binding proteins such as antibodies, that specifically interact with expression products of individual components of the candidate library are affixed to the chip in a logically ordered manner, i.e., in an array. In addition, any molecule with a specific affinity for either the sense or anti-sense sequence of the marker nucleotide sequence (depending on the design of the sample labeling), can be fixed to the array surface without loss of specific affinity for the marker and can be obtained and produced for array production, for example, proteins that specifically recognize the specific nucleic acid sequence of the marker, ribozymes, peptide nucleic acids (PNA), or other chemicals or molecules with specific affinity.

The techniques described herein, including array and microarray techniques, may be used to compare the gene expression profile of a biological sample from a patient of interest to the gene expression profile of a reference sample or other suitable control. The gene expression profile is determined by first extracting RNA from a biological sample of interest, such as from a patient diagnosed with an IBD. The RNA is then reverse transcribed into cDNA and labeled. In another embodiment, the cDNA may be transcribed into cRNA and labeled. The labeled cDNA or cRNA forms the target that may be hybridized to the array set comprising probes selected according the methods described herein. The reference sample obtained from a control patient is prepared in the same way. In one embodiment, both a test sample and reference sample may be used, the targets from each sample being differentially labeled (for example, with fluorophores having different excitation properties), and then combined and hybridized to the array under controlled conditions. In general, the labeled target and immobilized array sets are permitted, under appropriate conditions known to one of ordinary skill in the art, to hybridize such that the targets hybridize to complementary sequences on the arrays. After the array is washed with solutions of appropriately determined stringency to remove or reduce non-specific binding of labeled target, gene expression may be determined. The ratio of gene expression between the test sample and reference sample for a given gene determines the color and/or intensity of each spot, which can then be measured using standard techniques as known in the art. Analysis of the differential gene expression of a given array set provides an “expression profile” or “gene signature” for that array set. The expression profile is the pattern of gene expression produced by the experimental sample, wherein transcription of some genes are increased or decreased compared to the reference sample. Amplification methods using in vitro transcription may also be used to yield increased quantities of material to array where sample quantities are limited. In one embodiment, the Nugen Ovation amplification system may be incorporated into the protocol, as described below.

Commercially-produced, high-density arrays such as those manufactured by Affymetrix GeneChip (available from Affymetrix, Santa Clara, Calif.) containing synthesized oligonucleotides may be used with the methods disclosed herein. In one embodiment, the HGU133 Plus Version 2 Affymetrix GeneChip may be used to determine gene expression of an array sets comprising sequences listed in Tables 4-8 or the Sequence Listing.

In another embodiment, customized cDNA or oligonucleotide arrays may be manufactured by first selecting one or more array elements to be deposited on the array, selected from one or more sequences listed in Tables 4-8 or the Sequence Listing. Purified PCR products or other suitably derived oligonucleotides having the selected sequence may then be spotted or otherwise deposited onto a suitable matrix. The support may be selected from any suitable support known in the art, for example, microscope slides, glass, plastic or silicon chips, membranes such as nitrocellulose or paper, fibrous mesh arrangement, nylon filter arrays, glass-based arrays or the like. The array may be a chip array, a plate array, a bead array, a pin array, a membrane array, a solid surface array, a liquid array, an oligonucleotide array, a polynucleotide array, a cDNA array, a microfilter plate, a membrane or a chip. Where transparent surfaces such as microscope slides are used, the support provides the additional advantage of two-color fluorescent labeling with low inherent background fluorescence. The gene expression systems described above, such as arrays or microarrays, may be manufactured using any techniques known in the art, including, for example, printing with fine-pointed pins onto glass slides, photolithograpahy using dynamic micromirror devices, ink-jet printing, or electrochemistry on microelectrode arrays. Oligonucleotide adherence to the slide may be enhanced, for example, by treatment with polylysine or other cross-linking chemical coating or by any other method known in the art. The DNA or oligonucleotide may then be cross-linked by ultraviolet irradiation and denatured by exposure to either heat or alkali. The microarray may then be hybridized with labeled target derived from mRNA from one or more samples to be analyzed. For example, in one embodiment, cDNA or cRNA obtained from mRNA from colon samples derived from both a patient diagnosed with IBD and a healthy control sample is used. The samples may be labeled with different detectable labels such as, for example, fluorphores that exhibit different excitation properties. The samples may then be mixed and hybridized to a single microarray that is then scanned, allowing the visualization of up-regulated or down-regulated genes. The DualChip™ platform available from Eppendorf is an example of this type of array.

The probes affixed to the solid support in the gene expression system comprising the array elements may be a candidate library, a diagnostic agent, a diagnostic oligonucleotide set or a diagnostic probe set. In one embodiment of the present invention, the one or more array elements comprising the array set are selected from those sequences listed in Tables 4-8 or the Sequence Listing.

### Determination of Array Sets

A global pattern of gene expression in colon biopsies from Crohn's Disease (CD) patients at diagnosis (CDD), treated CD patients refractory to first line corticosteroid/6-MP therapy (chronic refractory, CDT), and healthy controls has been determined and is disclosed herein. cRNA was prepared from biopsies obtained from endoscopically affected segments, predominantly the ascending colon, with control biopsies obtained from matched segments in healthy patients. cRNA was labelled and then hybridized to the HGU133 Plus Version 2 Affymetrix GeneChip. RNA obtained from a pool of RNA from one normal colon specimen was labelled and hybridized to the GeneChip with each batch of new samples to serve as an internal control for batch to batch variability in signal intensity. Results were interpreted utilizing GeneSpring™ 7.3 Software (Silicon Genetics). Differentially expressed genes were identified by filtering levels of gene-specific signal intensity for statistically significant differences when grouped by clinical forms (e.g. healthy control versus CDD and healthy control versus CDT) using ANOVA, p values of <0.05 considered significant, without multiple testing correction and filtering for a fold-change expression level of at least 1.5-fold in the CDD versus normal and 2-fold for CDT versus normal. The overall gene expression profile was generated by gene tree hierarchical cluster analysis based on similarity of Pearson correlation, separation ratio 1, and minimal distance of 0.001.

An array set of 779 genes were identified. These genes, referred to as the Crohn's Disease Genomic Signature (Table 8) were differentially expressed in both CD colon at diagnosis and in chronic refractory disease, relative to healthy controls, with at least 1.5 fold difference in expression and significance level of at least 0.05. The global pattern of gene expression was substantially homogenous in the panel of chronic refractory patients, relative to a more heratogenous pattern in the CD patients at diagnosis, suggesting a distinct sub-set of CD patients that could be identified at diagnosis relative to their ultimate response to therapy. A cohort of CD patients having a known genomic signature was then prospectively followed.

From that cohort, responder patients and non-responder patients were identified. Treatment “responders” are defined as requiring one course of corticosteroids during the first year. Treatment “non-responders” are defined as requiring more than one course of corticosteroids, or anti-TNF, during the first year. The only clinical distinction between the responder and non-responder groups was the response to first line therapy, as they otherwise possessed similar age (12±1.2 vs 12±1.3, disease distribution, and clinical (Pediatric Crohn's Disease Activity Index (PCDAI): 40±9 vs 45±6) and histological (Crohn's Disease Histological Index of Severity (CDHIS): 6±1.8 vs 5±2) disease activity, respectively 70, 71. They also did not differ in the frequency of immunomodulator or mesalamine use.

Condition tree hierarchical cluster analysis using a distance correlation, in which the individual patients where grouped based upon similar patterns of gene expression and not pre-defined clinical subsets, has shown that most non-responders cluster together, with a pattern of gene expression intermediate between most responders and chronic refractory patients.

This gene set (the Crohn's Disease Genomic Signature, Table 8) was then reduced to smaller sets that can be used to distinguish responders from non-responders using the methods described herein. The smaller gene sets were identified via class prediction analysis using GeneSpring™ software, beginning with the CDGS gene set. The class prediction analysis used to arrive at the smaller gene sets is described in full below.

The smaller gene sets, referred to herein as “array sets” comprise the sequences disclosed in Tables 4-8 or the Sequence Listing. These array sets can be used to identify distinct sub-sets of CD patients at diagnosis, relative to their ultimate response to therapy. In particular, the gene sets, in one embodiment, may be used to determine whether a patient diagnosed with IBD may be classified as a “responder” or “non-responder,” thus permitting the clinician to predict the optimal course of therapy.

Thus, in one embodiment, gene expression methods can be used to define clinically meaningful sub-sets of IBD patients with respect to treatment response, using intestinal samples obtained at the time of diagnosis. Further, the CDGS and the K-nearest neighbors class prediction algorithm, using additional training and test sets derived from additional patient samples may be used to define novel array sets for predicting treatment response.

### Determination of a Gene Expression Profile

The present invention is related to methods of detecting gene expression using a gene expression system having one or more array elements wherein the array elements comprise one or more sequence that corresponds to sequence selected from those sequences listed in Tables 4-8 or the Sequence Listing, forming an array set. From the gene lists disclosed in Tables 4-8 and the Sequence Listing, it should be understood by one of ordinary skill in the art that standard methods of data analysis or using the disclosed methods (such as cluster analysis, K-nearest neighbors class prediction algorithms, or class prediction analysis using appropriately selected parameters) can be used to identify a smaller number of array elements, while still retaining the predictive characterisitics of the array sets disclosed herein. Non-limiting examples of data analysis that may be used are listed below.

In one embodiment, an array may be used to determine gene expression as described above. For example, PCR amplified inserts of cDNA clones may be applied to a substrate in a dense array. These cDNA may be selected from one or more of those sequences listed in Tables 4-8 or the Sequence Listing. In one embodiment, the array comprises a gene set further comprising one or more sequences listed in Table 4. In another embodiment, the array comprises an array set comprising one or more sequences listed in Table 5.

In another embodiment, the array (or gene expression system) comprises at least 2, 5, 10, 15, 20, 25, 30, 35, 40, 45, 50, 60, 70, 80, 90, 100, 150, 200, 250 or more different polynucleotide probes, each different probe capable of hybridizing to a different gene sequence listed in Table 6.

In another embodiment, the array (or gene expression system) comprises at least 2, 5, 10, 15, 20, 25, 30, 35, 40, 45, 50, 60, 70, 80, 90, 100, 150, 200, 250 or more different polynucleotide probes, each different probe capable of hybridizing to a different gene sequence listed in Table 7.

In another embodiment, the array (or gene expression system) comprising at least 2, 5, 10, 15, 20, 25, 30, 35, 40, 45, 50, 60, 70, 80, 90, 100, 150, 200, 250 or more different polynucleotide probes, each different probe capable of hybridizing to a different gene sequence listed in Table 8.

In one embodiment of the present invention, the array (or gene expression system) comprises a gene set further comprising from about 1 to about 1000 gene sequences, or about 200 to about 800 genes sequences, or about 20 to about 60 genes sequences, or about 10 to about 20 genes sequences, selected from the sequences listed in Tables 4-8 or the Sequence Listing.

In yet another embodiment, the selected genes include at least two groups of genes. The first group includes genes upregulated in inflammatory bowel disease compared to normal controls wherein the upregulated genes have IBD/Normal ratios of at least 2, 3, 4, 5, 10, or more. The second group includes genes downregulated in inflammatory bowel disease which have IBD/Normal ratios of no greater than 0.5, 0.333, 0.25, 0.2, 0.1, or less. Each group may include at least 1, 2, 4, 6, 8, 10, 12, 14, 16, 18, 20, or more genes.

It is also understood that each probe can correspond to one gene, or multiple probes can correspond to one gene, or both, or one probe can correspond to more than one gene. In some embodiments, DNA molecules are less than about any of the following lengths (in bases or base pairs): 10,000; 5,000; 2500; 2000; 1500; 1250; 1000; 750; 500; 300; 250; 200; 175; 150; 125; 100; 75; 50; 25; 10. In some embodiments, the DNA molecule is greater than about any of the following lengths (in bases or base pairs): 10; 15; 20; 25; 30; 40; 50; 60; 75; 100; 125; 150; 175; 200; 250; 300; 350; 400; 500; 750; 1000; 2000; 5000; 7500; 10000; 20000; 50000. Alternately, a DNA molecule can be any of a range of sizes having an upper limit of 10,000; 5,000; 2500; 2000; 1500; 1250; 1000; 750; 500; 300; 250; 200; 175; 150; 125; 100; 75; 50; 25; or 10 and an independently selected lower limit of 10; 15; 20; 25; 30; 40; 50; 60; 75; 100; 125; 150; 175; 200; 250; 300; 350; 400; 500; 750; 1000; 2000; 5000; 7500 wherein the lower limit is less than the upper limit.

Homologs and variants of the disclosed nucleic acid molecules in Tables 4-8 or the Sequence Listing may be used in the present invention. Homologs and variants of these nucleic acid molecules typically possess a relatively high degree of sequence identity when aligned using standard methods. Sequences suitable for use in the methods described herein have at least about 40-50, about 50-60, about 70-80, about 80-85, about 85-90, about 90-95 or about 95-100% sequence identity to the sequences disclosed herein.

The probes, immobilized on the selected substrate, are suitable for hybridization under conditions with appropriately determined stringency, such that targets binding non-specifically to the substrate or array elements are substantially removed. Appropriately labeled targets generated from mRNA are generated using any standard method as known in the art. For example, the targets may be cDNA targets generated through incorporation of fluorescent nucleotides by reverse transcription of RNA extracted from tissues of interest. Alternatively, biotin labeled targets may be used, such as using the method described herein. It should be clear that any suitable oligonucleotide-based target may be used. In another embodiment, suitably labeled cRNA targets may be used. Regardless of the type of target, the targets are such that the labeled targets applied to the chip hybridize to complementary probes on the array. After washing to minimize non-specific binding, the chip may be scanned by confocal laser microscopy or by any other suitable detection method known in the art, for example, a CCD camera. Quantification of hybridization at each spot in the array allows a determination of corresponding mRNA expression. With dual color fluorescence, separately labeled cDNA targets generated from two sources of RNA are hybridized pairwise to the array. The relative abundance of the transcripts from the two sources corresponding to each specified gene can then be determined simultaneously. (See Schena et al., Proc. Natl. Acad. Sci. USA 93(2): 106 149 (1996)). Microarray analysis can be performed by commercially available equipment, following manufacturer's protocols, such as by using the Affymetrix GenChip technology (for example, HGU133 Plus Version 2 Affymetrix GeneChip), or Incyte's microarray technology, or using any other methods as known in the art.

It is understood that for determination of a gene expression profile, variations in the disclosed sequences will still permit detection of gene expression. The degree of sequence identity required to detect gene expression varies depending on the length of the oligomer. For example, in a 60-mer, (an oligonucleotide with about 60 nucleotides), about 6 to about 8 random mutations or about 6 to about 8 random deletions in a 60-mer do not affect gene expression detection. Hughes, T R, et al. “Expression profiling using microarrays fabricated by an ink-jet oligonucleotide synthesizer. Nature Biotechnology, 19:343-347 (2001). As the length of the DNA sequence is increased, the number of mutations or deletions permitted while still allowing gene expression detection is increased.

As will be appreciated by those skilled in the art, the sequences of the present invention may contain sequencing errors. That is, there may be incorrect nucleotides, frameshifts, unknown nucleotides, or other types of sequencing errors in any of the sequences; however, the correct sequences will fall within the homology and stringency definitions herein.

### Additional Methods of Determining Gene Expression

The array sets disclosed herein may also be used to determine a gene expression profile such that a patient may be classified as a responder or a nonresponder any other techniques that measure gene expression. For example, the expression of genes disclosed in the array sets herein may be detected using RT-PCR methods or modified RT-PCR methods. In this embodiment, RT-PCR is used to detect gene expression of genes selected from one or more genes selected from the array sets listed in Tables 4-8 or the Sequence Listing.

Various methods using RT-PCR may be employed. For example, standard RT-PCR methods may be used. Using this method, well-known in the art, isolated RNA may be reverse transcribed using into cDNA using standard methods as known in the art. This cDNA is then exponentially amplified in a PCR reaction using standard PCR techniques. The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction. Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide is designed to detect nucleotide sequence located between the two PCR primers. The third oligonucleotide is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the third oligonucleotide in a template-dependent manner. The resultant fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data. TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data. To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin, although any other housekeeping gene or other gene established to be expressed at constant levels between comparison groups can be used.

Real time quantitative PCR techniques, which measure PCR product accumulation through a dual-labeled fluorigenic target (i.e., TaqMan® probe) may also be used with the methods disclosed herein to determine a gene expression profile. The Stratagene Brilliant SYBR Green QPCR reagent, available from 11011 N. Torrey Pines Road, La Jolla, Calif. 92037, may also be used. The SYBR® Green dye binds specifically to double-stranded PCR products, without the need for sequence-specific targets. Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996).

Alternatively, a modified RT-PCR method such as eXpress Profiling™ (XP) technology for high-throughput gene expression analysis, available from Althea Technologies, Inc. 11040 Roselle Street, San Diego, Calif. 92121 U.S.A. may be used to determine a gene expression profiles of a patient diagnosed with IBD. The gene expression analysis may be limited to one or more array sets as disclosed herein. This technology is described in U.S. Pat. No. 6,618,679, incorporated herein by reference. This technology uses a modified RT-PCR process that permits simultaneous, quantitative detection of expression levels of about 20 genes. This method may be complementary to or used in place of array technology or PCR and RT-PCR methods to determine or confirm a gene expression profile, for example, when classifying the status of a patient as a responder or non-responder.

Multiplex mRNA assays may also be used, for example, that described in Tian, et al., “Multiplex mRNA assay using Electrophoretic tags for high-throughput gene expression analysis,” Nucleic Acids Research 2004, Vol. 32, No. 16, published online Sep. 8, 2004 and Elnifro, et al. “Multiplex PCR: Optimization and Application in Diagnostic Virology,” Clinical Microbiology Reviews, October 2000, p. 559-570, both incorporated herein by reference. In multiplex CR, more than one target sequence can be amplified by including more than one pair of primers in the reation.

### Collection and Preparation of Sample

The methods disclosed herein employ a biological sample derived from patients diagnosed with an IBD such as UC or CD. The samples may include, for example, tissue samples obtained by biopsy of endoscopically affected colonic segments including the cecum/ascending, transverse/descending or sigmoid/rectum; small intestine; ileum; intestine; cell lysates; serum; or blood samples. Colon epithelia cells and lamina propria cells may be used for mRNA isolation. Control biopsies are obtained from the same source. Sample collection will depend on the target tissue or sample to be assayed.

Immediately after collection of a biological sample, the sample may be placed in a medium appropriate for storage of the sample such that degradation of mRNA is minimized and stored on ice. For example, a suitable medium for storage of sample until processing is RNALater®, available from Applied Biosystems, 850 Lincoln Centre Drive, Foster City Calif. 94404, U.S.A. Total RNA may then be prepared from a target sample using standard methods for RNA extraction known in the art and disclosed in standard textbooks of molecular biology, including Ausubel et al., Current Protocols of Molecular Biology, John Wiley and Sons (1997). For example, RNA isolation can be performed using purification kit, buffer set and protease from commercial manufacturers, such as Qiagen, according to the manufacturer's instructions. In one embodiment, total RNA is prepared utilizing the Qiagen RNeasy mini-column, available from QIAGEN Inc., 27220 Turnberry Lane Suite 200, Valencia, Calif. 91355. Other commercially available RNA isolation kits include MasterPure™ Complete DNA and RNA Purification Kit (EPICENTRE®, Madison, Wis.), or Paraffin Block RNA Isolation Kit (Ambion, Inc.). Total RNA from tissue samples may also be isolated using RNA Stat-60 (Tel-Test). RNA may also be prepared, for example, by cesium chloride density gradient centrifugation. RNA quality may then be assessed. RNA quality may be determined using, for example, the Agilent 2100 Bioanalyzer. Acceptable RNA samples have distinctive 18S and 28S Ribosomal RNA Bands and a 28S/18S ribosomal RNA ratio of about 1.5 to about 2.0.

In one embodiment, about 400 to about 500 nanograms of total RNA per sample is used to prepare labeled mRNA as targets. The RNA may be labeled using any methods known in the art, including for example, the TargetAmp 1-Round Aminoallyl-aRNA Amplification Kit available from Epicentre to prepare cRNA, following the manufacturer's instructions. The TargetAmp 1-Round Aminoallyl-aRNA Amplification Kit (Epicentre) is used to make double-stranded cDNA from total RNA. An in vitro transcription reaction creates cRNA target. Biotin-X-X-NHS (Epicentre) is used to label the aminoallyl-aRNA with biotin following the manufacturer's instructions. In one embodiment, the biotin-labeled cRNA target is then chemically fragmented and a hybridization cocktail is prepared and hybridized to a suitable array set immobilized on a suitable substrate. For example, the labeled cRNA may be hybridized to an Affymetrix Genechip Array (HGU133 Plus Version 2 Affymetrix GeneChip, available from Affymetrix, 3420 Central Expressway, Santa Clara, Calif. 95051). In this embodiment, the hybridization cocktail contains 0.034 ug/uL fragmented cRNA, 50 pM Control Oligonucleotide B2 (Affymetrix), 2OX Eukaryotic Hybridization Controls (1.5 pM bioB, 5 pM bioC, 25 pM bioD, 100 pM ere) (Affymetrix), 0.1 mg/mL Herring Sperm DNA (Promega), 0.5 mg/mL Acetylated BSA (Invitrogen), and IX Hybridization Buffer, though it should be understood that any suitable hybridization cocktail may be used.

In another embodiment, the total RNA may be used to prepare cDNA targets. The targets may be labeled using any suitable labels known in the art. The labeled cDNA targets may then be hybridized under suitable conditions to any array set or subset of an array set described herein, such that a gene expression profile may be obtained.

Normalization

Normalization is an adjustment made to microarray gene expression values to correct for potential bias or error introduced into an experiment. With respect to array-type analyses, such errors may be the result of unequal amounts of cDNA probe, differences in dye properties, differences in dye incorporation etc. Where appropriate, the present methods include the step of normalizing data to minimize the effects of bias or error. The type of normalization used will depend on the experimental design and the type of array being used. The type of normalization used will be understood by one of ordinary skill in the art.

Levels of Normalization

There may be two types of normalization levels used with the methods disclosed herein: “within slide” (this compensates, for example, for variation introduced by using different printing pins, unevenness in hybridization or, in the case of two channel arrays, differences in dye incorporation between the two samples) or “between slides,” which is sometimes referred to as “scaling” and permits comparison of results of different slides in an experiment, replicates, or different experiments.

Normalization Methods

Within slide normalization can be accomplished using local or global methods as known in the art. Local normalization methods include the use of “housekeeping genes” and “spikes” or “internal controls”. “Housekeeping” genes are genes which are known, or expected, not to change in expression level despite changes in disease state or phenotype or between groups of interest (such as between known non-responders and responders). For example, common housekeeping genes used to normalize data are those that encode for ubiquitin, actin and elongation factors. Where housekeeping genes are used, expression intensities on a slide are adjusted such that the housekeeping genes have the same intensity in all sample assays.

Normalization may also be achieved using spikes or internal controls that rely on RNA corresponding to particular probes on the microarray slide being added to each sample. These probes may be from a different species than the sample RNAs and optimally should not cross-hybridize to sample RNAs. For two channel arrays, the same amount of spike RNA is added to each sample prior to labeling and normalization is determined via measurement of the spiked features. Spikes can also be used to normalize spatially across a slide if the controls have been printed by each pin—the same controls on different parts of the slide should hybridize equally. Spikes may also be used to normalize between slides.

Reference samples may be any suitable reference sample or control as will be readily understood by one of skill in the art. For example, the reference sample may be selected from normal patients, “responder” patients, “non-responder” patients, or “chronic-refractory patients.” Normal patients are those not diagnosed with an IBD. “Responder” patients and “non-responder” patients are described above. “Chronic refractory” patients are patients with moderate to severe disease that require a second induction of remission using any drug. In one embodiment of the present invention, the control sample comprises cDNA from one or more patients that do not have an inflammatory bowel disease. In this embodiment, the cDNA of multiple normal samples are combined prior to labeling, and used as a control when determining gene expression of experimental samples. The data obtained from the gene expression analysis may then be normalized to the control cDNA.

A variety of global normalization methods may be used including, for example, linear regression. This method is suitable for two channel arrays and involves plotting the intensity values of one sample against the intensity values of the other sample. A regression line is then fitted to the data and the slope and intercept calculated. Intensity values in one channel are then adjusted so that the slope=1 and the intercept is 0. Linear regression can also be carried out using MA plots. These are plots of the log ratio between the Cy5 and Cy3 channel values against the average intensity of the two channels. Again regression lines are plotted and the normalized log ratios are calculated by subtracting the fitted value from the raw log ratio. In the alternative, lowess regression (locally weighted polynomial regression) may be used. This regression method again uses MA plots but is a non-linear regression method. This normalization method is suitable if the MA plots show that the intensity of gene expression is influencing the log ratio between the channels. Lowess essentially applies a large number of linear regressions using a sliding window of the data.

Yet another alternative method of normalization is “print tip normalization.” This is a form of spatial normalization that relies on the assumption that the majority of genes printed with individual print tips do not show differential expression. Either linear or non-linear regression can be used to normalize the data. Data from features printed by different print tips are normalized independently. This type of normalization is especially important when using single channel arrays.

Yet another method of normalization is “2D lowess normalization.” This form of spatial normalization uses a 2d polynomial lowess regression that is fitted to the data using a false color plot of log ratio or intensity as a function of the position of the feature on the array. Values are adjusted according to this polynomial. “Between slide normalization” enables you to compare results from different slides, whether they are two channel or single channel arrays.

Centering and scaling may also be used. This adjusts the distributions of the data (either of log ratios or signal intensity) on different slides such that the data is more similar. These adjustments ensure that the mean of the data distribution on each slide is zero and the standard deviation is 1. For each value on a slide, the mean of that slide is subtracted and the resulting value divided by the standard deviation of the slide. This ensures that the “spread” of the data is the same in each slide you are comparing.

Quantile normalization is yet another method that is particularly useful for comparing single channel arrays. Using this method, the data points in each slide are ranked from highest to lowest and the average computed for the highest values, second highest values and so on. The average value for that position is then assigned to each slide, i.e. the top ranked data point in each slide becomes the average of the original highest values and so on. This adjustment ensures that the data distributions on the different slides are identical.

Various tools for normalizing data are known in the art, and include GenePix, Excel, GEPAS, TMeV/MIDAS and R.

### Hybridization Techniques

Where array techniques are used to determine a gene expression profile, the targets must be hybridized to the array sets under suitable hybridization conditions using hybridization and wash solutions having appropriate stringency, such that labeled targets may hybridize to complementary probe sequences on the array. Washes of appropriate stringency are then used to remove non-specific binding of target to the array elements or substrate. Determination of appropriate stringency is within the ordinary skill of one skilled in the art.

In one embodiment of the present invention, the array set is that of the Affymetrix Genechip Array (HGU133 Plus Version 2 Affymetrix GeneChip, available from Affymetrix, 3420 Central Expressway, Santa Clara, Calif. 95051). In this embodiment, suitably labeled cRNA and hybridization cocktail are first prepared. In this embodiment, the hybridization cocktail contains about 0.034 ug/uL fragmented cRNA, about 50 pM Control Oligonucleotide B2 (available from Affymetrix), 2OX Eukaryotic Hybridization Controls (1.5 pM bioB, 5 pM bioC, 25 pM bioD, 100 pM ere) (available from Affymetrix), about 0.1 mg/mL Herring Sperm DNA (Promega), about 0.5 mg/mL Acetylated BSA (Invitrogen). The hybridization cocktail is heated to 99° C. for 5 minutes, to 45° C. for 5 minutes, and spun at maximum speed in a microcentrifuge for 5 minutes. The probe array is then filled with 200 uL of IX Hybridization Buffer (available from Affymetrix) and incubated at 45° C. for 10 minutes while rotating at 60 rpm. The IX Hybridization Buffer is removed and the probe array filled with 200 uL of the hybridization cocktail. The probe array is then incubated at 45° C. for about 16 hours in a hybridization oven rotating at 60 rpm.

The array is then washed and stained using any method as known in the art. In one embodiment, the Fluidics Station 450 (Affymetrix) and the fluidics protocol EukGE-WS2v4_{—}450 is used. This protocol comprises the steps of a first post-hybridization wash (10 cycles of 2 mixes/cycle with Affymetrix Wash Buffer A at 25° C.), a second post-hybridization wash (4 cycles of 15 mixes/cycle with Affymetrix Wash Buffer B at 50° C.), a first stain (staining the probe array for 10 minutes with Affymetrix Stain Cocktail 1 at 25° C.), a post-stain wash (10 cycles of 4 mixes/cycle with Affymetrix Wash Buffer A at 25° C.), a second stain (stain the probe array for 10 minutes with Stain Cocktail 2 at 25° C.), a third stain (stain the probe array for 10 minutes with Stain Cocktail 3 at 25° C.) and a final wash (15 cycles of 4 mixes/cycle with Wash Buffer A at 30° C. The holding temperature is 25° C.). All Wash Buffers and Stain Cocktails are those provided in the GeneChip® Hybridization, Wash and Stain Kit, Manufactured for Affymetrix, Inc., by Ambion, Inc., available from Affymetrix. In one embodiment, the stain used is R-Phycoerythrin Streptavidin, available from Molecular Probes. The antibody used is anti-streptavidin antibody (goat) biotinylated, available from Vector Laboratories.

Data Collection and Processing

When using an array to determine a gene expression profile, the data from the array must be obtained and processed. The data may then be used for any of the purposes set forth herein, such as to predict the outcome of a therapeutic treatment or to classify a patient as a responder or nonresponder.

Following appropriate hybridization and wash steps, the substrate containing the array set and hybridized target is scanned. Data is then collected and may be saved as both an image and a text file. Precise databases and tracking of files should be maintained regarding the location of the array elements on the substrates. Information on the location and names of genes should also be maintained. The files may then be imported to software programs that perform image analysis and statistical analysis functions.

The gene expression profile of a patient of interest is then determined from the collected data. This may be done using any standard method that permits qualitative or quantitative measurements as described herein. Appropriate statistical methods may then be used to predict the significance of the variation in the gene expression profile, and the probability that the patient's gene expression profile is within the category of non-responder or responder. For example, in one embodiment, the data may be collected, then analyzed such that a class determination may be made (i.e., categorizing a patient as a responder or nonresponder) using a class prediction algorithm and GeneSpring™ software as described below.

Expression patterns can be evaluated by qualitative and/or quantitative measures. Qualitative methods detect differences in expression that classify expression into distinct modes without providing significant information regarding quantitative aspects of expression. For example, a technique can be described as a qualitative technique if it detects the presence or absence of expression of a candidate nucleotide sequence, i.e., an on/off pattern of expression. Alternatively, a qualitative technique measures the presence (and/or absence) of different alleles, or variants, of a gene product.

In contrast, some methods provide data that characterize expression in a quantitative manner. That is, the methods relate expression on a numerical scale, e.g., a scale of 0-5, a scale of 1-10, a scale of +−+++, from grade 1 to grade 5, a grade from a to z, or the like. It will be understood that the numerical, and symbolic examples provided are arbitrary, and that any graduated scale (or any symbolic representation of a graduated scale) can be employed in the context of the present invention to describe quantitative differences in nucleotide sequence expression. Typically, such methods yield information corresponding to a relative increase or decrease in expression.

Any method that yields either quantitative or qualitative expression data is suitable for evaluating expression. In some cases, e.g., when multiple methods are employed to determine expression patterns for a plurality of candidate nucleotide sequences, the recovered data, e.g., the expression profile, for the nucleotide sequences is a combination of quantitative and qualitative data.

In some applications, expression of the plurality of candidate nucleotide sequences is evaluated sequentially. This is typically the case for methods that can be characterized as low- to moderate-throughput. In contrast, as the throughput of the elected assay increases, expression for the plurality of candidate nucleotide sequences in a sample or multiple samples is assayed simultaneously. Again, the methods (and throughput) are largely determined by the individual practitioner, although, typically, it is preferable to employ methods that permit rapid, e.g. automated or partially automated, preparation and detection, on a scale that is time-efficient and cost-effective.

It is understood that the preceding discussion is directed at both the assessment of expression of the members of candidate libraries and to the assessment of the expression of members of diagnostic nucleotide sets.

Many techniques have been applied to the problem of making sense of large amounts of gene expression data. Cluster analysis techniques (e.g., K-Means), self-organizing maps (SOM), principal components analysis (PCA), and other analysis techniques are all widely available in packaged software used in correlating this type of gene expression data.

Class Prediction

In one embodiment, the data obtained may be analyzed using a class prediction algorithm to predict whether a subject is a non-responder or a responder, as defined above. Class prediction is a supervised learning method in which the algorithm learns from samples with known class membership (the training set) and establishes a prediction rule to classify new samples (the test set). Class prediction consists of several steps. The first is feature selection, a process by which genes within a defined gene set are scored for their ability to distinguish between classes (responders and non-responders) in the training set. Genes may be selected for uses as predictors, by individual examination and ranking based on the power of the gene to discriminate responders from non-responders. Genes may then be scored on the basis of the best prediction point for responders or non-responders. The score function is the negative natural logarithm of the p-value for a hypergeometric test of predicted versus actual group membership for responder versus non-responder. A combined list for responders and non-responders for the most discriminating genes may then be produced, up to the number of predictor genes specified by the user. The Golub method may then be used to test each gene considered for the predictor gene set for its ability to discriminate responders from non-responders using a signal-to-noise ratio. Genes with the highest scores may then be kept for subsequent calculations. A subset of genes with high predictive strength may then used in class prediction, with cross validation performed using the known groups from the training set. The K-nearest neighbors approach may be used to classify training set samples during cross validation, and to classify test set samples once the predictive rule had been established. In this system, each sample is classified by finding the K-nearest neighboring training set samples (where K is the number of neighbors defined by the user) plotted based in Euclidean space over normalized expression intensity for each of the genes in the predictor set. For example, a predictive gene set of twenty members may be selected using four nearest neighbors. Depending on the number of samples available, the k value may vary. The class membership of the selected number of nearest neighbors to each sample is enumerated and p-values computed to determine the likelihood of seeing at least the observed number of neighbors from each class relative to the whole training set by chance in a K-sized neighborhood. With this method, the confidence in class prediction is best determined by the ratio of the smallest p-value and the second smallest p-value, termed the decision cut-off p-value. If it is lower, the test sample is classified as the class corresponding to the smallest p-value. If it is higher, a prediction is not made. In one embodiment, a decision cut-off p-value ratio of about 0.5 may be used. Cross validation in GeneSpring may then be then done by a drop-one-out algorithm, in which the accuracy of the prediction rule is tested. This approach removes one sample from the training set and uses it as a test sample. By predicting the class of a given sample only after it is removed from the training set, the rule makes unbiased prediction of the sample class. Once performance of the predictive rule has been optimized in this fashion, it may be tested using additional samples.

### Cluster Analysis

Cluster analysis is a loose term covering many different algorithms for grouping data. Clustering can be divided into two main types: top-down and bottom-up. Top-down clustering starts with a given number of clusters or classes and proceeds to partition the data into these classes. Bottom-up clustering starts by grouping data at the lowest level and builds larger groups by bringing the smaller groups together at the next highest level.

K-Means is an example of top-down clustering. K-means groups data into K number of best-fit clusters. Before using the algorithm, the user defines the number of clusters that are to be used to classify the data (K clusters). The algorithm randomly assigns centers to each cluster and then partitions the nearest data into clusters with those centers. The algorithm then iteratively finds new centers by averaging over the data in the cluster and reassigning data to new clusters as the centers change. The analysis iteratively continues until the centers no longer move (Sherlock, G., Current Opinion in Immunology, 12:201, 2000).

Tree clustering is an example of bottom-up clustering. Tree clustering joins data together by assigning nearest pairs as leaves on the tree. When all pairs have been assigned (often according to either information-theoretical criteria or regression methods), the algorithm progresses up to the next level joining the two nearest groups from the prior level as one group. Thus, the number and size of the clusters depends on the level. Often, the fewer clusters, the larger each cluster will be. The stoppage criteria for such algorithms varies, but often is determined by an analysis of the similarity of the members inside the cluster compared to the difference across the clusters.

Self-organizing maps (SOMs) are competitive neural networks that group input data into nearest neighbors (Torkkola, K., et al., Information Sciences, 139:79, 2001; Toronen, P., et al., FEBS Letters, 451:142 146, 1999). As data is presented to the neural network, neurons whose weights currently are capable of capturing that data (the winner neuron) are updated toward the input. Updating the weights, or training the neural net, shifts the recognition space of each neuron toward a center of similar data. SOMs are similar to K-means with the added constraint that all centers are on a 1 or 2 dimensional manifold (i.e., the feature space is mapped into a 1 or 2 dimensional array, where new neighborhoods are formed). In SOM, the number of neurons is chosen to be much larger than the possible number of the clusters. It is hoped that the clusters of trained neurons will provide a good estimation of the number of the neurons. In many cases, however, a number of small clusters are formed around the larger clusters, and there is no practical way of distinguishing such smaller clusters from, or of merging them into, the larger clusters. In addition, there is no guarantee that the resulting clusters of genes actually exhibit statistically independent expression profiles. Thus, the members of two different clusters may exhibit similar patterns of gene expression.

Principal component analysis (PCA), although not a clustering technique in its nature (Jolliffe, I. T., Principal Component Analysis, New York: Springer-Verlag, 1986) can also be used for clustering (Yeung, K. Y., et al., Bioinformatics, 17:763, 2001). PCA is a stepwise analysis that attempts to create a new component axis at each step that contains most of the variation seen for the data. Thus, the first component explains the first most important basis for the variation in the data, the second component explains the second most important basis for the variation in the data, the third component the third most important basis, and so on. PCA projects the data into a new space spanned by the principal components. Each successive principal component is selected to be orthogonal to the previous ones, and to capture the maximum information that is not already present in the previous components. The principal components are therefore linear combinations (or eigenarrays) of the original data. These principal components are the classes of data in the new coordinate generated by PCA. If the data is highly non-correlated, then the number of significant principal components can be as high as the number of original data values. If, as in the case of DNA microarray experiments, the data is expected to correlate among groups, than the data should be described by a set of components which is fewer than the full complement of data points.

A variety of systems known in the art may be used for image analysis and compiling the data. For example, where the mRNA is labeled with a fluorescent tag, and fluorescence imaging system (such as the microarray processor commercially available from AFFYMETRIX®, Santa Clara, Calif.) may be used to capture, and quantify the extent of hybridization at each address. Or, in the case where the mRNA is radioactive, the array may be exposed to X-ray film and a photographic image made. Once the data is collected, it may be compiled to quantify the extent of hybridization at each address as for example, using software to convert the measured signal to a numerical value.

Any publicly available imaging software may be used. Examples include BioDiscovery (ImaGene), Axon Instruments (GenePix Pro 6.0), EisenLab—Stanford University (ScanAlyze), Spotfinder (TIGR), Imaxia (ArrayFox), F-Scan (Analytical Biostatistics Section—NIH), MicroDiscovery (GeneSpotter), CLONDIAG (IconoClust), Koada Technology (Koadarray), Vigene Tech (Micro Vigene), Nonlinear Dynamics (Phoretix), CSIRO Mathematical and Information Sciences (SPOT) Niles Scientific (SpotReader).

Any commercially available data analysis software may also be used. Examples include, BRB Array Tools (Biometric Research Branch—NCI), caGEDA (University of Pittsburgh), Cleaver 1.0 (Stanford Biomedical Informatics), ChipSC2C (Peterson Lab—Baylor College of Medicine), Cluster (Eisen Lab—Stanford/UC Berkeley), DNA-Chip Analyzer (dChip) (Wong Laboratory—Harvard University), Expression Profiler (European Bioinformatics Institute), FuzzyK (Eisen Lab—Stanford/UC Berkeley), GeneCluster 2.0 (Broad Institute), GenePattern (Broad Institute), GeneXPress (Stanford University), Genesis (Alexander Sturn—Graz University of Technology), GEPAS (Spanish National Cancer Center), GLR (University of Utah), GQL (Max Planck Institute for Molecular Genetics), INCLUSive (Katholieke Universiteit Leuven), Maple Tree (Eisen Lab—Stanford/UC Berkeley) MeV (TIGR) MIDAS (TIGR), Onto-Tools (Sorin Draghici—Wayne State University), Short Time-series Expression Miner (Carnegie Mellon University), Significance Analysis of Microarrays (Rob Tibshirani—Stanford University), SNOMAD (Johns Hopkins Schools of Medicine and Public Health), SparseLOGREG (Shevade & Keerthi—National University of Singapore), SuperPC Microarrays (Rob Tibshirani—Stanford University), Table View (University of Minnesota), TreeView (Eisen Lab—Stanford/UC Berkeley), Venn Mapper (Universitais Medisch Centrum Rotterdam), Applied Maths (GeneMaths XT), Array Genetics (AffyMate), Axon Instruments (Acuity 4.0) BioDiscovery (GeneSight), BioSieve (ExpressionSieve), CytoGenomics (SilicoCyte), Microarray Data Analysis (GeneSifter), MediaCybernetics (ArrayPro Analyzer), Microarray Fuzzy Clustering (BioRainbow), Molmine (J-Express Pro), Optimal Design (Array Miner), Partek (Partek Pro) Predictive Patterns Software (GeneLinker), Promoter Extractor (BioRainbow) SAS Microarray Silicon Genetics (GeneSpring), Spotfire (Spotfire), Strand Genomics (Avadis) Vialogy Corp.

It should also be understood that confounding factors may exist in individual subjects that may affect the ability of a given gene set to predict responders versus non-responders. These cofounding variables include variation in medications, such as cases in which concurrent 6-MP with infliximab overcomes the adverse effects of an unfavorable FasL polymorphism on response, the CARD15 genotype status, or the location of the biopsy, due to variation of gene expression along the colon. To account for this variation, outliers may be identified, and subsequently determined whether the outliers may be accounted for by variations in medication use, CARD 15 genotype, or the location of the colon biopsy.

Kits

In an additional aspect, the present invention provides kits embodying the methods, compositions, and systems for analysis of gene expression as described herein. Kits of the present invention may comprise one or more of the following: a) at least one pair of universal primers; b) at least one pair of target-specific primers, wherein the primers are specific to one or more sequences listed in Tables 4-8 or the sequence listing; c) at least one pair of reference gene-specific primers; and d) one or more amplification reaction enzymes, reagents, or buffers. The universal primers provided in the kit may include labeled primers. The target-specific primers may vary from kit to kit, depending upon the specified target gene(s) to be investigated, and may also be labeled. Exemplary reference gene-specific primers (e.g., target-specific primers for directing transcription of one or more reference genes) include, but are not limited to, primers for β-actin, cyclophilin, GAPDH, and various rRNA molecules.

The kits of the invention optionally include one or more preselected primer sets that are specific for the genes to be amplified. The preselected primer sets optionally comprise one or more labeled nucleic acid primers, contained in suitable receptacles or containers. Exemplary labels include, but are not limited to, a fluorophore, a dye, a radiolabel, an enzyme tag, etc., that is linked to a nucleic acid primer itself.

In addition, one or more materials and/or reagents required for preparing a biological sample for gene expression analysis are optionally included in the kit. Furthermore, optionally included in the kits are one or more enzymes suitable for amplifying nucleic acids, including various polymerases (RT, Taq, etc.), one or more deoxynucleotides, and buffers to provide the necessary reaction mixture for amplification.

In one embodiment of the invention, the kits are employed for analyzing gene expression patterns using mRNA as the starting template. The mRNA template may be presented as either total cellular RNA or isolated mRNA. In other embodiments, the methods and kits described in the present invention allow quantification of other products of gene expression, including tRNA, rRNA, or other transcription products. In still further embodiments, other types of nucleic acids may serve as template in the assay, including genomic or extragenomic DNA, viral RNA or DNA, or nucleic acid polymers generated by non-replicative or artificial mechanism, including PNA or RNA/DNA copolymers.

Optionally, the kits of the present invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided.

Array Sets 1-5 are listed below in Tables 4-8.

TABLE 4
Array Set 1
IBD Patients Gene Expression Relative to Healthy Controls (p < 0.05)

| Affymetrix | GenBank | | Fold |
|---|---|---|---|
| Number | Accession No. | Gene Name | Change |
| NM_001099_at | NM_001099 | ACPP | 1.837 |
| NM_001150_at | NM_001150 | ANPEP | 0.285 |
| NM_004900_at | NM_004900 | APOBEC3B | 0.352 |
| NM_001169_at | NM_001169 | AQP8 | 0.263 |
| NM_006829_at | NM_006829 | C10orf116 | 0.405 |
| NM_001276_at | NM_001276 | CHI3L1 | 5.374 |
| NM_001855_at | NM_001855 | COL15A1 | 1.981 |
| NM_001845_at | NM_001845 | COL4A1 | 1.81 |
| NM_000093_at | NM_000093 | COL5A1 | 1.664 |
| NM_001849_at | NM_001849 | COL6A2 | 2.069 |
| NM_001511_at | NM_001511 | CXCL1 | 6.583 |
| NM_002994_at | NM_002994 | CXCL5 | 4.465 |
| NM_002993_at | NM_002993 | CXCL6 | 5.086 |
| NM_000772_at | NM_000772 | CYP2C18 | 0.436 |
| NM_013974_at | NM_013974 | DDAH2 | 0.529 |
| NM_139160_at | NM_139160 | DEPDC7 | 0.436 |
| NM_207581_at | NM_207581 | DUOXA2 | 2.53 |
| NM_001425_at | NM_001425 | EMP3 | 2.027 |
| NM_001249_at | NM_001249 | ENTPD5 | 0.439 |
| NM_016594_at | NM_016594 | FKBP11 | 2.848 |
| NM_002023_at | NM_002023 | FMOD | 1.724 |
| NM_212474_at | NM_212474 | FN1 | 1.867 |
| NM_212475_at | NM_212475 | FN1 | 1.867 |
| NM_212478_at | NM_212478 | FN1 | 1.867 |
| NM_212476_at | NM_212476 | FN1 | 1.866 |
| NM_212482_at | NM_212482 | FN1 | 1.865 |
| NM_002026_at | NM_002026 | FN1 | 1.858 |
| NM_001491_at | NM_001491 | GCNT2 | 0.536 |
| NM_145655_at | NM_145655 | GCNT2 | 0.535 |
| NM_145649_at | NM_145649 | GCNT2 | 0.535 |
| NM_024307_at | NM_024307 | GDPD3 | 0.565 |
| NM_001031718_at | NM_001031718 | GDPD3 | 0.564 |
| NM_014905_at | NM_014905 | GLS | 0.546 |
| NM_004297_at | NM_004297 | GNA14 | 2.074 |
| NM_198447_at | NM_198447 | GOLT1A | 0.455 |
| NM_000558_at | NM_000558 | HBA1 | 2.245 |
| NM_000517_at | NM_000517 | HBA2 | 1.903 |
| NM_002153_at | NM_002153 | HSD17B2 | 0.309 |
| NM_000198_at | NM_000198 | HSD3B2 | 0.151 |
| NM_006855_at | NM_006855 | KDELR3 | 2.278 |
| NM_005564_at | NM_005564 | LCN2 | 2.882 |
| NM_012318_at | NM_012318 | LETM1 | 0.653 |
| NM_005925_at | NM_005925 | MEP1B | 0.122 |
| NM_152637_at | NM_152637 | METTL7B | 0.483 |
| NM_002422_at | NM_002422 | MMP3 | 10.8 |
| NM_138928_at | NM_138928 | MOCS1 | 0.522 |
| NM_005943_at | NM_005943 | MOCS1 | 0.521 |
| NM_005942_at | NM_005942 | MOCS1 | 0.521 |
| NM_145015_at | NM_145015 | MRGPRF | 1.8 |
| NM_015419_at | NM_015419 | MXRA5 | 1.931 |
| NM_153292_at | NM_153292 | NOS2A | 2.877 |
| NM_000625_at | NM_000625 | NOS2A | 2.874 |
| NM_153240_at | NM_153240 | NPHP3 | 0.584 |
| NM_002593_at | NM_002593 | PCOLCE | 1.979 |
| NM_000439_at | NM_000439 | PCSK1 | 3.694 |
| NM_000440_at | NM_000440 | PDE6A | 0.397 |
| NM_007350_at | NM_007350 | PHLDA1 | 1.807 |
| NM_015900_at | NM_015900 | PLA1A | 2.31 |
| NM_145202_at | NM_145202 | PRAP1 | 0.291 |
| NM_002742_at | NM_002742 | PRKD1 | 1.548 |
| NM_058179_at | NM_058179 | PSAT1 | 2.612 |
| NM_021154_at | NM_021154 | PSAT1 | 2.603 |
| NM_002841_at | NM_002841 | PTPRG | 1.743 |
| NM_016339_at | NM_016339 | RAPGEFL1 | 0.495 |
| NM_003469_at | NM_003469 | SCG2 | 1.909 |
| NM_000295_at | NM_000295 | SERPINA1 | 1.917 |
| NM_001002236_at | NM_001002236 | SERPINA1 | 1.916 |
| NM_001002235_at | NM_001002235 | SERPINA1 | 1.916 |
| NM_016276_at | NM_016276 | SGK2 | 0.399 |
| NM_170693_at | NM_170693 | SGK2 | 0.399 |
| NM_003051_at | NM_003051 | SLC16A1 | 0.41 |
| NM_004695_at | NM_004695 | SLC16A5 | 0.487 |
| NM_005415_at | NM_005415 | SLC20A1 | 0.57 |
| NM_007231_at | NM_007231 | SLC6A14 | 8.39 |
| NM_014464_at | NM_014464 | TINAG | 0.498 |
| NM_015444_at | NM_015444 | TMEM158 | 2.778 |
| NM_024873_at | NM_024873 | TNIP3 | 1.655 |
| NM_178234_at | NM_178234 | TUSC3 | 2.835 |
| NM_006765_at | NM_006765 | TUSC3 | 2.831 |
| NM_057179_at | NM_057179 | TWIST2 | 1.572 |
| NM_004666_at | NM_004666 | VNN1 | 2.398 |
| NM_025079_at | NM_025079 | ZC3H12A | 1.905 |
| NM_174945_at | NM_174945 | ZNF575 | 0.554 |
| NM_001008397_at | NM_001008397 | | 3.388 |
| NM_016459_at | NM_016459 | | 2.341 |
| NM_001018060_at | NM_001018060 | | 0.496 |
| NM_138342_at | NM_138342 | | 0.475 |
| NM_178859_at | NM_178859 | | 0.474 |
| NM_144704_at | NM_144704 | | 0.467 |
| XM_930288_at | XM_930288 | | 0.464 |
| XM_943650_at | XM_943650 | | 0.463 |
| XM_943644_at | XM_943644 | | 0.463 |
| XM_938362_at | XM_938362 | | 0.463 |
| XM_943655_at | XM_943655 | | 0.463 |
| XM_934563_at | XM_934563 | | 0.463 |
| XM_934567_at | XM_934567 | | 0.463 |
| XM_934562_at | XM_934562 | | 0.462 |
| XM_943653_at | XM_943653 | | 0.46 |
| XM_934566_at | XM_934566 | | 0.459 |
| NM_152672_at | NM_152672 | | 0.17 |

TABLE 5
Array Set 2 (n = 20, derived using the k-nearest neighbors algorithm)
Gene Expression of Responder (R) and NonResponder (NR) Patients with IBD

| Affymetrix | GenBank | Gene | Fold | Fold | Predictive |
|---|---|---|---|---|---|
| Number | Accession No. | Name | Change (R) | Change (NR) | Strength |
| NM_001169_at | NM_001169 | AQP8 | 0.6 | 0.1 | 4.2 |
| NM_000093_at | NM_000093 | COL5A1 | 0.7 | 1.2 | 4.2 |
| NM_002023_at | NM_002023 | FMOD | 1 | 1.9 | 5.8 |
| NM_024307_at | NM_024307 | GDPD3 | 1.5 | 0.8 | 4.2 |
| NM_001031718_at | NM_001031718 | GDPD3 | 1.5 | 0.8 | 4.2 |
| NM_004297_at | NM_004297 | GNA14 | 1 | 1.7 | 5.8 |
| NM_198447_at | NM_198447 | GOLT1A | 1.1 | 0.6 | 4.2 |
| NM_012318_at | NM_012318 | LETM1 | 1.3 | 0.8 | 4.2 |
| NM_153292_at | NM_153292 | NOS2A | 2.4 | 4.3 | 4.2 |
| NM_000625_at | NM_000625 | NOS2A | 2.4 | 4.3 | 4.2 |
| NM_000439_at | NM_000439 | PCSK1 | 0.8 | 5.6 | 5.8 |
| NM_016339_at | NM_016339 | RAPGEFL1 | 0.9 | 0.5 | 4.2 |
| NM_000295_at | NM_000295 | SERPINA1 | 1.5 | 3.1 | 4.2 |
| NM_001002236_at | NM_001002236 | SERPINA1 | 1.5 | 3.1 | 4.2 |
| NM_001002235_at | NM_001002235 | SERPINA1 | 1.5 | 3.1 | 4.2 |
| NM_016276_at | NM_016276 | SGK2 | 0.9 | 0.3 | 4.2 |
| NM_170693_at | NM_170693 | SGK2 | 0.9 | 0.3 | 4.2 |
| NM_015444_at | NM_015444 | TMEM158 | 1.3 | 3.9 | 4.2 |
| NM_001008397_at | NM_001008397 | | 2 | 4.4 | 5.8 |
| NM_178859_at | NM_178859 | | 1 | 0.4 | 4.2 |

TABLE 6
Array Set 3 (n = 24, derived using ANOVA)
Gene Expression of Responder (R) and Non-Responder (NR) Patients with IBD

| Affymetrix | GenBank | Gene | Fold | Fold | |
|---|---|---|---|---|---|
| Number | Accession No. | Name | Change (R) | Change (NR) | P Value |
| NM_001150_at | NM_001150 | ANPEP | 1.1 | 0.3 | 0.0356 |
| NM_006829_at | NM_006829 | C10orf116 | 0.8 | 0.4 | 0.0213 |
| NM_000093_at | NM_000093 | COL5A1 | 0.7 | 1.2 | 0.0188 |
| NM_001249_at | NM_001249 | ENTPD5 | 1 | 0.5 | 0.0323 |
| NM_001491_at | NM_001491 | GCNT2 | 1 | 0.6 | 0.00966 |
| NM_145655_at | NM_145655 | GCNT2 | 1 | 0.6 | 0.00973 |
| NM_145649_at | NM_145649 | GCNT2 | 1 | 0.6 | 0.0105 |
| NM_024307_at | NM_024307 | GDPD3 | 1.5 | 0.8 | 0.0203 |
| NM_001031718_at | NM_001031718 | GDPD3 | 1.5 | 0.8 | 0.0205 |
| NM_198447_at | NM_198447 | GOLT1A | 1.1 | 0.6 | 0.0244 |
| NM_006855_at | NM_006855 | KDELR3 | 2 | 3.3 | 0.0173 |
| NM_005564_at | NM_005564 | LCN2 | 6.6 | 17 | 0.0136 |
| NM_005925_at | NM_005925 | MEP1B | 0.7 | 0.3 | 0.0156 |
| NM_153292_at | NM_153292 | NOS2A | 2.4 | 4.3 | 0.00602 |
| NM_000625_at | NM_000625 | NOS2A | 2.4 | 4.3 | 0.00609 |
| NM_000439_at | NM_000439 | PCSK1 | 0.8 | 5.6 | 0.00188 |
| NM_145202_at | NM_145202 | PRAP1 | 0.7 | 0.3 | 0.00805 |
| NM_003051_at | NM_003051 | SLC16A1 | 0.6 | 0.3 | 0.0297 |
| NM_015444_at | NM_015444 | TMEM158 | 1.3 | 3.9 | 0.0305 |
| NM_004666_at | NM_004666 | VNN1 | 1.5 | 5 | 0.016 |
| NM_025079_at | NM_025079 | ZC3H12A | 1.5 | 2.7 | 0.0383 |
| NM_001008397_at | NM_001008397 | | 2 | 4.4 | 0.00181 |
| NM_178859_at | NM_178859 | | 1 | 0.4 | 0.0228 |
| NM_152672_at | NM_152672 | | 0.7 | .01 | 0.0172 |

TABLE 7
Array Set 4
Colon Gene Set Differentially Expressed Between Responders (R) and Non-
Responders (NR).

| | | | | Non- |
|---|---|---|---|---|
| | | Predictive | Responder | responder |
| Gene | Function | Strength | Expression | Expression |
| DDAH2 | nitric oxide generation | 1.5 | 0.7 | 0.4 |
| EMP1 | adhesion | 1.2 | 0.7 | 0.3 |
| ENTPD5 | catabolism of extracellular nucleotides | 1.1 | 0.8 | 0.4 |
| GCNT2 | CHO antigen processing | 1.0 | 0.8 | 0.4 |
| GLS | phosphate-activated glutaminase | 1.3 | 0.8 | 0.5 |
| GNA14 | guanine nucleotide binding protein | 1.2 | 1.4 | 2.6 |
| KDELR3 | ER protein sorting | 1.1 | 1.9 | 3.6 |
| LCN2 | PMN granule protein | 1.4 | 4.2 | 12.3 |
| LOC49386 | oxidative stress response | 1.2 | 2.4 | 5.2 |
| MYH10 | myosin, heavy polypeptide 10, non- | 1.0 | 1.3 | 2.7 |
| | muscle | | | |
| NOS2A | nitric oxide synthase 2A | 1.0 | 2 | 3.3 |
| PCSK1 | proprotein convertase | 1.4 | 1 | 6.6 |
| PRAP1 | proline-rich acidic protein 1 | 1.0 | 0.6 | 0.2 |
| SAA2 | APR | 1.0 | 1.3 | 4 |
| SLC20A1 | solute carrier family 20 (phosphate | 1.2 | 0.7 | 0.3 |
| | transporter), member 1 | | | |
| TUSC3 | tumor suppressor | 1.3 | 1.3 | 3.1 |
| TWSG1 | twisted gastrulation homolog 1 | 1.0 | 1.2 | 2.2 |
| VNN1 | oxidative stress response | 1.2 | 2 | 11.9 |

TABLE 8
Array Set 5
Crohn's Disease Genomic Signature

| | Affymetrix | GenBank | | |
|---|---|---|---|---|
| Gene Name | Number | Accession No. | Fold Change | Sequence ID No. |
| ACADS | NM_000017_at | NM_000017 | 0.557 | 1. |
| ACOT4 | NM_152331_at | NM_152331 | 0.537 | 2. |
| ACOT8 | NM_183386_at | NM_183386 | 0.651 | 3. |
| ACOT8 | NM_005469_at | NM_005469 | 0.626 | 4. |
| ACOT8 | NM_183385_at | NM_183385 | 0.626 | 5. |
| ACPP | NM_001099_at | NM_001099 | 1.837 | 6. |
| ACSL4 | NM_004458_at | NM_004458 | 2.084 | 7. |
| ACSL4 | NM_022977_at | NM_022977 | 2.076 | 8. |
| ACVR1 | NM_001105_at | NM_001105 | 1.763 | 9. |
| ADAM19 | NM_033274_at | NM_033274 | 1.904 | 10. |
| ADAM9 | NM_003816_at | NM_003816 | 1.726 | 11. |
| ADAM9 | NM_001005845_at | NM_001005845 | 1.725 | 12. |
| ADAMTS1 | NM_006988_at | NM_006988 | 2.112 | 13. |
| ADCY3 | NM_004036_at | NM_004036 | 1.54 | 14. |
| ADM | NM_001124_at | NM_001124 | 2.344 | 15. |
| AGA | NM_000027_at | NM_000027 | 1.564 | 16. |
| AGBL2 | NM_024783_at | NM_024783 | 0.557 | 17. |
| AGT | NM_000029_at | NM_000029 | 2.162 | 18. |
| AHSA2 | NM_152392_at | NM_152392 | 0.632 | 19. |
| AK1 | NM_000476_at | NM_000476 | 0.58 | 20. |
| AKAP2 | NM_001004065_at | NM_001004065 | 1.769 | 21. |
| AKR7A3 | NM_012067_at | NM_012067 | 0.608 | 22. |
| ALS2CL | NM_182775_at | NM_182775 | 0.613 | 23. |
| ALS2CL | NM_147129_at | NM_147129 | 0.613 | 24. |
| AMICA1 | NM_153206_at | NM_153206 | 1.77 | 25. |
| ANPEP | NM_001150_at | NM_001150 | 0.285 | 26. |
| ANTXR1 | NM_032208_at | NM_032208 | 1.503 | 27. |
| ANXA1 | NM_000700_at | NM_000700 | 2.056 | 28. |
| ANXA3 | NM_005139_at | NM_005139 | 1.687 | 29. |
| ANXA5 | NM_001154_at | NM_001154 | 1.725 | 30. |
| APCDD1 | NM_153000_at | NM_153000 | 2.807 | 31. |
| APOBEC3B | NM_004900_at | NM_004900 | 0.352 | 32. |
| APOBEC3G | NM_021822_at | NM_021822 | 2.302 | 33. |
| APOL1 | NM_003661_at | NM_003661 | 1.916 | 34. |
| APOL1 | NM_145343_at | NM_145343 | 1.913 | 35. |
| APOL3 | NM_014349_at | NM_014349 | 2.036 | 36. |
| APOL3 | NM_030644_at | NM_030644 | 2.034 | 37. |
| APOL3 | NM_145639_at | NM_145639 | 2.032 | 38. |
| APOL3 | NM_145641_at | NM_145641 | 2.032 | 39. |
| APOL3 | NM_145640_at | NM_145640 | 2.032 | 40. |
| APOL3 | NM_145642_at | NM_145642 | 2.029 | 41. |
| AQP8 | NM_001169_at | NM_001169 | 0.263 | 42. |
| ARFGAP3 | NM_014570_at | NM_014570 | 2.138 | 43. |
| ARHGEF3 | NM_019555_at | NM_019555 | 1.729 | 44. |
| ARMCX2 | NM_177949_at | NM_177949 | 1.673 | 45. |
| ARMCX2 | NM_014782_at | NM_014782 | 1.672 | 46. |
| ASPH | NM_032468_at | NM_032468 | 1.505 | 47. |
| ASPHD2 | NM_020437_at | NM_020437 | 1.717 | 48. |
| ATP2C1 | NM_001001486_at | NM_001001486 | 1.514 | 49. |
| ATP2C1 | NM_001001485_at | NM_001001485 | 1.513 | 50. |
| ATP2C1 | NM_001001487_at | NM_001001487 | 1.513 | 51. |
| AVIL | NM_006576_at | NM_006576 | 0.553 | 52. |
| AYTL2 | NM_024830_at | NM_024830 | 1.73 | 53. |
| B4GALNT2 | NM_153446_at | NM_153446 | 0.582 | 54. |
| BAG2 | NM_004282_at | NM_004282 | 1.802 | 55. |
| BAIAP2L2 | NM_025045_at | NM_025045 | 0.57 | 56. |
| BMP6 | NM_001718_at | NM_001718 | 1.918 | 57. |
| BNIP3 | NM_004052_at | NM_004052 | 2.227 | 58. |
| BSG | NM_198589_at | NM_198589 | 0.662 | 59. |
| BSG | NM_198590_at | NM_198590 | 0.662 | 60. |
| BSG | NM_198591_at | NM_198591 | 0.662 | 61. |
| BSG | NM_001728_at | NM_001728 | 0.661 | 62. |
| BTN3A2 | NM_007047_at | NM_007047 | 1.603 | 63. |
| C10orf116 | NM_006829_at | NM_006829 | 0.405 | 64. |
| C12orf28 | NM_182530_at | NM_182530 | 0.567 | 65. |
| C14orf29 | NM_181814_at | NM_181814 | 0.546 | 66. |
| C14orf29 | NM_181533_at | NM_181533 | 0.542 | 67. |
| C16orf14 | NM_138418_at | NM_138418 | 0.524 | 68. |
| C1orf116 | NM_023938_at | NM_023938 | 0.529 | 69. |
| C1orf188 | NM_173795_at | NM_173795 | 0.618 | 70. |
| C1orf38 | NM_001039477_at | NM_001039477 | 2.147 | 71. |
| C1orf38 | NM_004848_at | NM_004848 | 2.144 | 72. |
| C1QB | NM_000491_at | NM_000491 | 3.16 | 73. |
| C1R | NM_001733_at | NM_001733 | 2.263 | 74. |
| C1S | NM_001734_at | NM_001734 | 2.359 | 75. |
| C1S | NM_201442_at | NM_201442 | 2.358 | 76. |
| C20orf100 | NM_032883_at | NM_032883 | 1.981 | 77. |
| C20orf56 | NR_001558_at | NR_001558 | 1.788 | 78. |
| C4A | NM_007293_at | NM_007293 | 2.775 | 79. |
| C4B | NM_001002029_at | NM_001002029 | 2.774 | 80. |
| C4BPA | NM_000715_at | NM_000715 | 2.228 | 81. |
| C4BPB | NM_001017366_at | NM_001017366 | 1.876 | 82. |
| C4BPB | NM_001017367_at | NM_001017367 | 1.874 | 83. |
| C4BPB | NM_000716_at | NM_000716 | 1.871 | 84. |
| C4BPB | NM_001017364_at | NM_001017364 | 1.863 | 85. |
| C4BPB | NM_001017365_at | NM_001017365 | 1.863 | 86. |
| C5orf14 | NM_024715_at | NM_024715 | 1.612 | 87. |
| C5orf20 | NM_130848_at | NM_130848 | 1.688 | 88. |
| C6orf136 | NM_145029_at | NM_145029 | 0.586 | 89. |
| C7orf10 | NM_024728_at | NM_024728 | 0.547 | 90. |
| C9orf72 | NM_018325_at | NM_018325 | 1.535 | 91. |
| CALCRL | NM_005795_at | NM_005795 | 1.725 | 92. |
| CALD1 | NM_033138_at | NM_033138 | 1.832 | 93. |
| CALD1 | NM_004342_at | NM_004342 | 1.831 | 94. |
| CALD1 | NM_033157_at | NM_033157 | 1.83 | 95. |
| CALD1 | NM_033139_at | NM_033139 | 1.717 | 96. |
| CALD1 | NM_033140_at | NM_033140 | 1.716 | 97. |
| CAPN3 | NM_173090_at | NM_173090 | 0.622 | 98. |
| CAPN3 | NM_173089_at | NM_173089 | 0.622 | 99. |
| CAPN3 | NM_173087_at | NM_173087 | 0.618 | 100. |
| CAPN3 | NM_173088_at | NM_173088 | 0.618 | 101. |
| CAPN3 | NM_212464_at | NM_212464 | 0.618 | 102. |
| CAPN3 | NM_000070_at | NM_000070 | 0.617 | 103. |
| CAPN3 | NM_212465_at | NM_212465 | 0.617 | 104. |
| CAPN3 | NM_212467_at | NM_212467 | 0.617 | 105. |
| CAPN3 | NM_024344_at | NM_024344 | 0.617 | 106. |
| CARD15 | NM_022162_at | NM_022162 | 2.375 | 107. |
| CARD6 | NM_032587_at | NM_032587 | 1.708 | 108. |
| CBFA2T3 | NM_175931_at | NM_175931 | 1.525 | 109. |
| CBFA2T3 | NM_005187_at | NM_005187 | 1.517 | 110. |
| CBR3 | NM_001236_at | NM_001236 | 1.581 | 111. |
| CCLI1 | NM_002986_at | NM_002986 | 3.005 | 112. |
| CCL2 | NM_002982_at | NM_002982 | 3.652 | 113. |
| CCL20 | NM_004591_at | NM_004591 | 2.091 | 114. |
| CCL8 | NM_005623_at | NM_005623 | 3.269 | 115. |
| CCPG1 | NM_004748_at | NM_004748 | 1.792 | 116. |
| CCPG1 | NM_020739_at | NM_020739 | 1.791 | 117. |
| CD14 | NM_001040021_at | NM_001040021 | 1.742 | 118. |
| CD14 | NM_000591_at | NM_000591 | 1.742 | 119. |
| CD300A | NM_007261_at | NM_007261 | 1.816 | 120. |
| CD300LF | NM_139018_at | NM_139018 | 1.893 | 121. |
| CD38 | NM_001775_at | NM_001775 | 2.373 | 122. |
| CD74 | NM_004355_at | NM_004355 | 2.276 | 123. |
| CD74 | NM_001025158_at | NM_001025158 | 2.276 | 124. |
| CD74 | NM_001025159_at | NM_001025159 | 2.264 | 125. |
| CD81 | NM_004356_at | NM_004356 | 1.686 | 126. |
| CD86 | NM_175862_at | NM_175862 | 2.043 | 127. |
| CD86 | NM_006889_at | NM_006889 | 2.028 | 128. |
| CDH11 | NM_001797_at | NM_001797 | 2.714 | 129. |
| CDH13 | NM_001257_at | NM_001257 | 1.787 | 130. |
| CECR1 | NM_017424_at | NM_017424 | 2.429 | 131. |
| CECR1 | NM_177405_at | NM_177405 | 2.428 | 132. |
| CFI | NM_000204_at | NM_000204 | 2.091 | 133. |
| CFL2 | NM_138638_at | NM_138638 | 1.528 | 134. |
| CGNL1 | NM_032866_at | NM_032866 | 1.633 | 135. |
| CH25H | NM_003956_at | NM_003956 | 3.471 | 136. |
| CHI3L1 | NM_001276_at | NM_001276 | 5.374 | 137. |
| CHKB | NM_152253_at | NM_152253 | 0.615 | 138. |
| CHST11 | NM_018413_at | NM_018413 | 1.726 | 139. |
| CHST13 | NM_152889_at | NM_152889 | 0.55 | 140. |
| CHST2 | NM_004267_at | NM_004267 | 2.509 | 141. |
| CHSY1 | NM_014918_at | NM_014918 | 1.686 | 142. |
| CLDN15 | NM_014343_at | NM_014343 | 0.641 | 143. |
| CLEC10A | NM_006344_at | NM_006344 | 2.546 | 144. |
| CLEC10A | NM_182906_at | NM_182906 | 2.539 | 145. |
| CLEC4A | NM_194447_at | NM_194447 | 2.471 | 146. |
| CLEC4A | NM_194448_at | NM_194448 | 2.47 | 147. |
| CLEC4A | NM_194450_at | NM_194450 | 2.412 | 148. |
| CLEC4A | NM_016184_at | NM_016184 | 2.41 | 149. |
| CLEC7A | NM_197950_at | NM_197950 | 1.976 | 150. |
| CLEC7A | NM_197954_at | NM_197954 | 1.908 | 151. |
| CLEC7A | NM_022570_at | NM_022570 | 1.826 | 152. |
| CLEC7A | NM_197947_at | NM_197947 | 1.826 | 153. |
| CLEC7A | NM_197949_at | NM_197949 | 1.825 | 154. |
| CLEC7A | NM_197948_at | NM_197948 | 1.823 | 155. |
| CMAH | NR_002174_at | NR_002174 | 1.654 | 156. |
| CMKOR1 | NM_020311_at | NM_020311 | 2.054 | 157. |
| COL15A1 | NM_001855_at | NM_001855 | 1.981 | 158. |
| COL1A2 | NM_000089_at | NM_000089 | 2.069 | 159. |
| COL3A1 | NM_000090_at | NM_000090 | 1.8 | 160. |
| COL4A1 | NM_001845_at | NM_001845 | 1.81 | 161. |
| COL5A1 | NM_000093_at | NM_000093 | 1.664 | 162. |
| COL5A2 | NM_000393_at | NM_000393 | 1.853 | 163. |
| COL6A2 | NM_001849_at | NM_001849 | 2.069 | 164. |
| COL6A3 | NM_004369_at | NM_004369 | 2.388 | 165. |
| COL6A3 | NM_057164_at | NM_057164 | 2.386 | 166. |
| COL6A3 | NM_057165_at | NM_057165 | 2.386 | 167. |
| COL6A3 | NM_057167_at | NM_057167 | 2.386 | 168. |
| COL6A3 | NM_057166_at | NM_057166 | 2.385 | 169. |
| COLEC11 | NM_024027_at | NM_024027 | 0.652 | 170. |
| CPA3 | NM_001870_at | NM_001870 | 5.314 | 171. |
| CPT1B | NM_152247_at | NM_152247 | 0.615 | 172. |
| CPT1B | NM_152246_at | NM_152246 | 0.57 | 173. |
| CPT1B | NM_004377_at | NM_004377 | 0.561 | 174. |
| CPT1B | NM_152245_at | NM_152245 | 0.56 | 175. |
| CPVL | NM_019029_at | NM_019029 | 4.537 | 176. |
| CPVL | NM_031311_at | NM_031311 | 4.536 | 177. |
| CRISPLD2 | NM_031476_at | NM_031476 | 2.115 | 178. |
| CRYL1 | NM_015974_at | NM_015974 | 0.566 | 179. |
| CSF1R | NM_005211_at | NM_005211 | 2.035 | 180. |
| CSF2RA | NM_172247_at | NM_172247 | 1.958 | 181. |
| CSF2RA | NM_172245_at | NM_172245 | 1.94 | 182. |
| CSF2RA | NM_006140_at | NM_006140 | 1.931 | 183. |
| CSF2RA | NM_172246_at | NM_172246 | 1.897 | 184. |
| CSF2RA | NM_172248_at | NM_172248 | 1.621 | 185. |
| CSPG2 | NM_004385_at | NM_004385 | 2.834 | 186. |
| CTGF | NM_001901_at | NM_001901 | 2.021 | 187. |
| CTHRC1 | NM_138455_at | NM_138455 | 2.914 | 188. |
| CTSC | NM_148170_at | NM_148170 | 2.289 | 189. |
| CTSC | NM_001814_at | NM_001814 | 1.985 | 190. |
| CTSK | NM_000396_at | NM_000396 | 1.901 | 191. |
| CTSO | NM_001334_at | NM_001334 | 1.533 | 192. |
| CX3CR1 | NM_001337_at | NM_001337 | 2.373 | 193. |
| CXCL1 | NM_001511_at | NM_001511 | 6.583 | 194. |
| CXCL10 | NM_001565_at | NM_001565 | 4.095 | 195. |
| CXCL11 | NM_005409_at | NM_005409 | 5.809 | 196. |
| CXCL12 | NM_000609_at | NM_000609 | 1.673 | 197. |
| CXCL2 | NM_002089_at | NM_002089 | 3.404 | 198. |
| CXCL3 | NM_002090_at | NM_002090 | 3.087 | 199. |
| CXCL5 | NM_002994_at | NM_002994 | 4.465 | 200. |
| CXCL6 | NM_002993_at | NM_002993 | 5.086 | 201. |
| CXCL9 | NM_002416_at | NM_002416 | 6.414 | 202. |
| CYP27A1 | NM_000784_at | NM_000784 | 0.66 | 203. |
| CYP2C18 | NM_000772_at | NM_000772 | 0.436 | 204. |
| CYP2C9 | NM_000771_at | NM_000771 | 0.285 | 205. |
| CYP4F12 | NM_023944_at | NM_023944 | 0.55 | 206. |
| CYP4F2 | NM_001082_at | NM_001082 | 0.499 | 207. |
| CYP4X1 | NM_178033_at | NM_178033 | 1.569 | 208. |
| CYR61 | NM_001554_at | NM_001554 | 3.992 | 209. |
| DDAH2 | NM_013974_at | NM_013974 | 0.529 | 210. |
| DEGS1 | NM_144780_at | NM_144780 | 1.84 | 211. |
| DEGS1 | NM_003676_at | NM_003676 | 1.836 | 212. |
| DEPDC7 | NM_139160_at | NM_139160 | 0.436 | 213. |
| DFNA5 | NM_004403_at | NM_004403 | 1.573 | 214. |
| DNAJC12 | NM_021800_at | NM_021800 | 1.865 | 215. |
| DOCK4 | NM_014705_at | NM_014705 | 2.058 | 216. |
| DQX1 | NM_133637_at | NM_133637 | 0.471 | 217. |
| DUOX2 | NM_014080_at | NM_014080 | 14.74 | 218. |
| DUOXA2 | NM_207581_at | NM_207581 | 2.53 | 219. |
| DUSP4 | NM_001394_at | NM_001394 | 1.507 | 220. |
| DUSP4 | NM_057158_at | NM_057158 | 1.504 | 221. |
| EAF2 | NM_018456_at | NM_018456 | 2.034 | 222. |
| EDN1 | NM_001955_at | NM_001955 | 0.517 | 223. |
| EGR2 | NM_000399_at | NM_000399 | 1.842 | 224. |
| EIF2AK4 | NM_001013703_at | NM_001013703 | 1.536 | 225. |
| ELL2 | NM_012081_at | NM_012081 | 2.324 | 226. |
| ELL3 | NM_025165_at | NM_025165 | 0.615 | 227. |
| EML1 | NM_001008707_at | NM_001008707 | 1.501 | 228. |
| EMP3 | NM_001425_at | NM_001425 | 2.027 | 229. |
| EMR2 | NM_152920_at | NM_152920 | 2.119 | 230. |
| EMR2 | NM_152918_at | NM_152918 | 2.117 | 231. |
| EMR2 | NM_152919_at | NM_152919 | 2.117 | 232. |
| EMR2 | NM_013447_at | NM_013447 | 2.11 | 233. |
| EMR2 | NM_152917_at | NM_152917 | 2.11 | 234. |
| EMR2 | NM_152921_at | NM_152921 | 2.108 | 235. |
| EMR2 | NM_152916_at | NM_152916 | 2.107 | 236. |
| ENTPD1 | NM_001776_at | NM_001776 | 2.514 | 237. |
| ENTPD5 | NM_001249_at | NM_001249 | 0.439 | 238. |
| ERO1LB | NM_019891_at | NM_019891 | 1.711 | 239. |
| ETNK1 | NM_018638_at | NM_018638 | 0.455 | 240. |
| EVA1 | NM_144765_at | NM_144765 | 0.628 | 241. |
| F2R | NM_001992_at | NM_001992 | 1.887 | 242. |
| FADS1 | NM_013402_at | NM_013402 | 1.925 | 243. |
| FAM46C | NM_017709_at | NM_017709 | 2.071 | 244. |
| FAM73B | NM_032809_at | NM_032809 | 0.626 | 245. |
| FAM89A | NM_198552_at | NM_198552 | 1.539 | 246. |
| FAM92A1 | XM_943013_at | XM_943013 | 1.54 | 247. |
| FBLN1 | NM_001996_at | NM_001996 | 1.886 | 248. |
| FBLN5 | NM_006329_at | NM_006329 | 1.725 | 249. |
| FBN1 | NM_000138_at | NM_000138 | 1.79 | 250. |
| FBXO6 | NM_018438_at | NM_018438 | 1.888 | 251. |
| FCER1G | NM_004106_at | NM_004106 | 3.497 | 252. |
| FCGR3B | NM_000570_at | NM_000570 | 3.507 | 253. |
| FGR | NM_005248_at | NM_005248 | 2.047 | 254. |
| FKBP11 | NM_016594_at | NM_016594 | 2.848 | 255. |
| FMOD | NM_002023_at | NM_002023 | 1.724 | 256. |
| FN1 | NM_212474_at | NM_212474 | 1.867 | 257. |
| FN1 | NM_212475_at | NM_212475 | 1.867 | 258. |
| FN1 | NM_212478_at | NM_212478 | 1.867 | 259. |
| FN1 | NM_212476_at | NM_212476 | 1.866 | 260. |
| FN1 | NM_212482_at | NM_212482 | 1.865 | 261. |
| FN1 | NM_002026_at | NM_002026 | 1.858 | 262. |
| FOXF2 | NM_001452_at | NM_001452 | 1.989 | 263. |
| FSTL1 | NM_007085_at | NM_007085 | 1.749 | 264. |
| FUT8 | NM_178157_at | NM_178157 | 1.651 | 265. |
| FUT8 | NM_178154_at | NM_178154 | 1.651 | 266. |
| FUT8 | NM_178156_at | NM_178156 | 1.65 | 267. |
| FUT8 | NM_178155_at | NM_178155 | 1.65 | 268. |
| FUT8 | NM_004480_at | NM_004480 | 1.648 | 269. |
| FZD2 | NM_001466_at | NM_001466 | 1.963 | 270. |
| FZD3 | NM_017412_at | NM_017412 | 1.713 | 271. |
| GALNT5 | NM_014568_at | NM_014568 | 1.655 | 272. |
| GBP1 | NM_002053_at | NM_002053 | 2.671 | 273. |
| GBP5 | NM_052942_at | NM_052942 | 4.008 | 274. |
| GCNT2 | NM_001491_at | NM_001491 | 0.536 | 275. |
| GCNT2 | NM_145655_at | NM_145655 | 0.535 | 276. |
| GCNT2 | NM_145649_at | NM_145649 | 0.535 | 277. |
| GDPD3 | NM_024307_at | NM_024307 | 0.565 | 278. |
| GDPD3 | NM_001031718_at | NM_001031718 | 0.564 | 279. |
| GEM | NM_005261_at | NM_005261 | 1.669 | 280. |
| GEM | NM_181702_at | NM_181702 | 1.666 | 281. |
| GGT1 | NM_005265_at | NM_005265 | 0.457 | 282. |
| GGT1 | NM_001032364_at | NM_001032364 | 0.455 | 283. |
| GGT1 | NM_013430_at | NM_013430 | 0.455 | 284. |
| GGT1 | NM_001032365_at | NM_001032365 | 0.455 | 285. |
| GGT2 | NM_002058_at | NM_002058 | 0.447 | 286. |
| GGTL4 | NM_080839_at | NM_080839 | 0.439 | 287. |
| GGTL4 | NM_199127_at | NM_199127 | 0.438 | 288. |
| GGTLA4 | NM_178311_at | NM_178311 | 0.616 | 289. |
| GGTLA4 | NM_178312_at | NM_178312 | 0.615 | 290. |
| GGTLA4 | NM_080920_at | NM_080920 | 0.613 | 291. |
| GLCCI1 | NM_138426_at | NM_138426 | 1.816 | 292. |
| GLS | NM_014905_at | NM_014905 | 0.546 | 293. |
| GNA14 | NM_004297_at | NM_004297 | 2.074 | 294. |
| GNA15 | NM_002068_at | NM_002068 | 2.036 | 295. |
| GOLGA2L1 | NM_017600_at | NM_017600 | 0.624 | 296. |
| GOLT1A | NM_198447_at | NM_198447 | 0.455 | 297. |
| GPR109B | NM_006018_at | NM_006018 | 4.219 | 298. |
| GPR124 | NM_032777_at | NM_032777 | 1.577 | 299. |
| GPR137B | NM_003272_at | NM_003272 | 2.101 | 300. |
| GPR37 | NM_005302_at | NM_005302 | 1.771 | 301. |
| GSTA1 | NM_145740_at | NM_145740 | 0.242 | 302. |
| HAS2 | NM_005328_at | NM_005328 | 2.046 | 303. |
| HAVCR1 | NM_012206_at | NM_012206 | 0.654 | 304. |
| HBA1 | NM_000558_at | NM_000558 | 2.245 | 305. |
| HBA2 | NM_000517_at | NM_000517 | 1.903 | 306. |
| HBB | NM_000518_at | NM_000518 | 2.965 | 307. |
| HCK | NM_002110_at | NM_002110 | 2.218 | 308. |
| HDC | NM_002112_at | NM_002112 | 1.593 | 309. |
| HLA-DPA1 | NM_033554_at | NM_033554 | 2.78 | 310. |
| HLA-DQB1 | NM_002123_at | NM_002123 | 1.764 | 311. |
| HLA-DRA | NM_019111_at | NM_019111 | 2.398 | 312. |
| HLA-DRB1 | NM_002124_at | NM_002124 | 1.843 | 313. |
| HLA-DRB3 | NM_022555_at | NM_022555 | 1.858 | 314. |
| HLA-DRB6 | NR_001298_at | NR_001298 | 1.753 | 315. |
| HNRPL | NM_001533_at | NM_001533 | 0.559 | 316. |
| HNRPL | NM_001005335_at | NM_001005335 | 0.558 | 317. |
| HOXB5 | NM_002147_at | NM_002147 | 0.599 | 318. |
| HOXB6 | NM_018952_at | NM_018952 | 0.643 | 319. |
| HSD11B1 | NM_181755_at | NM_181755 | 2.776 | 320. |
| HSD11B1 | NM_005525_at | NM_005525 | 2.764 | 321. |
| HSD17B2 | NM_002153_at | NM_002153 | 0.309 | 322. |
| HSD17B6 | NM_003725_at | NM_003725 | 1.629 | 323. |
| HSD3B1 | NM_000862_at | NM_000862 | 0.522 | 324. |
| HSD3B2 | NM_000198_at | NM_000198 | 0.151 | 325. |
| HSPB1 | NM_001540_at | NM_001540 | 0.6 | 326. |
| HTRA1 | NM_002775_at | NM_002775 | 1.513 | 327. |
| ICAM1 | NM_000201_at | NM_000201 | 1.76 | 328. |
| IFI30 | NM_006332_at | NM_006332 | 2.189 | 329. |
| IGFBP7 | NM_001553_at | NM_001553 | 1.677 | 330. |
| IGSF6 | NM_005849_at | NM_005849 | 2.666 | 331. |
| IL10RA | NM_001558_at | NM_001558 | 2.181 | 332. |
| IL12RB1 | NM_153701_at | NM_153701 | 1.642 | 333. |
| IL1B | NM_000576_at | NM_000576 | 3.534 | 334. |
| IL2RB | NM_000878_at | NM_000878 | 2.002 | 335. |
| IL8 | NM_000584_at | NM_000584 | 4.708 | 336. |
| IL8RB | NM_001557_at | NM_001557 | 2.26 | 337. |
| INDO | NM_002164_at | NM_002164 | 4.548 | 338. |
| IRF4 | NM_002460_at | NM_002460 | 1.717 | 339. |
| IRS1 | NM_005544_at | NM_005544 | 2.14 | 340. |
| ISL1 | NM_002202_at | NM_002202 | 1.904 | 341. |
| ITGB2 | NM_000211_at | NM_000211 | 2.335 | 342. |
| ITPKA | NM_002220_at | NM_002220 | 0.497 | 343. |
| JAK2 | NM_004972_at | NM_004972 | 1.703 | 344. |
| KCTD12 | NM_138444_at | NM_138444 | 1.666 | 345. |
| KCTD14 | NM_023930_at | NM_023930 | 1.547 | 346. |
| KDELR3 | NM_006855_at | NM_006855 | 2.278 | 347. |
| KIAA0125 | NM_014792_at | NM_014792 | 2.828 | 348. |
| KIAA0367 | NM_015225_at | NM_015225 | 1.593 | 349. |
| KIT | NM_000222_at | NM_000222 | 2.567 | 350. |
| KLF8 | NM_007250_at | NM_007250 | 0.451 | 351. |
| KLKB1 | NM_000892_at | NM_000892 | 0.556 | 352. |
| KRT12 | NM_000223_at | NM_000223 | 0.268 | 353. |
| LAMC1 | NM_002293_at | NM_002293 | 1.723 | 354. |
| LAX1 | NM_017773_at | NM_017773 | 2.229 | 355. |
| LCN2 | NM_005564_at | NM_005564 | 2.882 | 356. |
| LCP2 | NM_005565_at | NM_005565 | 2.458 | 357. |
| LDHD | NM_153486_at | NM_153486 | 0.448 | 358. |
| LDHD | NM_194436_at | NM_194436 | 0.447 | 359. |
| LETM1 | NM_012318_at | NM_012318 | 0.653 | 360. |
| LHFP | NM_005780_at | NM_005780 | 1.94 | 361. |
| LIMS1 | NM_004987_at | NM_004987 | 1.52 | 362. |
| LIPC | NM_000236_at | NM_000236 | 0.56 | 363. |
| LOXL1 | NM_005576_at | NM_005576 | 2.022 | 364. |
| LPHN2 | NM_012302_at | NM_012302 | 2.057 | 365. |
| LRRK2 | NM_198578_at | NM_198578 | 1.948 | 366. |
| LUM | NM_002345_at | NM_002345 | 3.195 | 367. |
| LYN | NM_002350_at | NM_002350 | 1.634 | 368. |
| LYSMD2 | NM_153374_at | NM_153374 | 1.732 | 369. |
| MAGEH1 | NM_014061_at | NM_014061 | 1.757 | 370. |
| MAP3K5 | NM_005923_at | NM_005923 | 1.523 | 371. |
| MARVELD3 | NM_001017967_at | NM_001017967 | 0.568 | 372. |
| MCOLN2 | NM_153259_at | NM_153259 | 0.42 | 373. |
| MDS1 | NM_004991_at | NM_004991 | 0.533 | 374. |
| ME3 | NM_006680_at | NM_006680 | 0.528 | 375. |
| ME3 | NM_001014811_at | NM_001014811 | 0.527 | 376. |
| MEOX1 | NM_004527_at | NM_004527 | 1.857 | 377. |
| MEOX1 | NM_001040002_at | NM_001040002 | 1.85 | 378. |
| MEOX1 | NM_013999_at | NM_013999 | 1.842 | 379. |
| MEP1B | NM_005925_at | NM_005925 | 0.122 | 380. |
| METTL7B | NM_152637_at | NM_152637 | 0.483 | 381. |
| MFAP4 | NM_002404_at | NM_002404 | 1.954 | 382. |
| MICAL3 | XM_943874_at | XM_943874 | 0.611 | 383. |
| MITF | NM_006722_at | NM_006722 | 1.779 | 384. |
| MITF | NM_198178_at | NM_198178 | 1.777 | 385. |
| MITF | NM_198177_at | NM_198177 | 1.777 | 386. |
| MITF | NM_198158_at | NM_198158 | 1.773 | 387. |
| MITF | NM_198159_at | NM_198159 | 1.773 | 388. |
| MITF | NM_000248_at | NM_000248 | 1.772 | 389. |
| MMP1 | NM_002421_at | NM_002421 | 6.11 | 390. |
| MMP10 | NM_002425_at | NM_002425 | 3.311 | 391. |
| MMP12 | NM_002426_at | NM_002426 | 4.267 | 392. |
| MMP2 | NM_004530_at | NM_004530 | 2.249 | 393. |
| MMP3 | NM_002422_at | NM_002422 | 10.8 | 394. |
| MMP7 | NM_002423_at | NM_002423 | 2.139 | 395. |
| MNDA | NM_002432_at | NM_002432 | 4.425 | 396. |
| MOCS1 | NM_138928_at | NM_138928 | 0.522 | 397. |
| MOCS1 | NM_005943_at | NM_005943 | 0.521 | 398. |
| MOCS1 | NM_005942_at | NM_005942 | 0.521 | 399. |
| MOGAT2 | NM_025098_at | NM_025098 | 0.57 | 400. |
| MORC4 | NM_024657_at | NM_024657 | 1.662 | 401. |
| MPST | NM_001013440_at | NM_001013440 | 0.613 | 402. |
| MPST | NM_021126_at | NM_021126 | 0.612 | 403. |
| MPST | NM_001013436_at | NM_001013436 | 0.612 | 404. |
| MRGPRF | NM_145015_at | NM_145015 | 1.8 | 405. |
| MS4A2 | NM_000139_at | NM_000139 | 1.934 | 406. |
| MTHFD2 | NM_006636_at | NM_006636 | 1.928 | 407. |
| MTHFD2 | NM_001040409_at | NM_001040409 | 1.927 | 408. |
| MTMR11 | NM_181873_at | NM_181873 | 0.579 | 409. |
| MXRA5 | NM_015419_at | NM_015419 | 1.931 | 410. |
| MYBL1 | XM_938064_at | XM_938064 | 0.659 | 411. |
| MYBL1 | XM_034274_at | XM_034274 | 0.658 | 412. |
| MYH10 | NM_005964_at | NM_005964 | 1.904 | 413. |
| MYL5 | NM_002477_at | NM_002477 | 0.441 | 414. |
| NCF2 | NM_000433_at | NM_000433 | 2.801 | 415. |
| NEIL1 | NM_024608_at | NM_024608 | 0.59 | 416. |
| NID1 | NM_002508_at | NM_002508 | 1.617 | 417. |
| NID2 | NM_007361_at | NM_007361 | 1.774 | 418. |
| NINJ2 | NM_016533_at | NM_016533 | 1.502 | 419. |
| NMU | NM_006681_at | NM_006681 | 1.518 | 420. |
| NOS2A | NM_153292_at | NM_153292 | 2.877 | 421. |
| NOS2A | NM_000625_at | NM_000625 | 2.874 | 422. |
| NOX1 | NM_013955_at | NM_013955 | 1.73 | 423. |
| NPHP3 | NM_153240_at | NM_153240 | 0.584 | 424. |
| NQO2 | NM_000904_at | NM_000904 | 2.038 | 425. |
| NR4A2 | NM_173172_at | NM_173172 | 1.758 | 426. |
| NR4A2 | NM_173171_at | NM_173171 | 1.758 | 427. |
| NR4A2 | NM_173173_at | NM_173173 | 1.758 | 428. |
| NR4A2 | NM_006186_at | NM_006186 | 1.757 | 429. |
| NUCB2 | NM_005013_at | NM_005013 | 2.403 | 430. |
| OASL | NM_003733_at | NM_003733 | 0.439 | 431. |
| OASL | NM_198213_at | NM_198213 | 0.439 | 432. |
| OLFM1 | NM_006334_at | NM_006334 | 1.65 | 433. |
| OLFML3 | NM_020190_at | NM_020190 | 2.075 | 434. |
| OSMR | NM_003999_at | NM_003999 | 1.561 | 435. |
| OTUD3 | XM_375697_at | XM_375697 | 0.666 | 436. |
| P2RY13 | NM_176894_at | NM_176894 | 3.812 | 437. |
| P2RY13 | NM_023914_at | NM_023914 | 3.811 | 438. |
| PAM | NM_000919_at | NM_000919 | 1.713 | 439. |
| PAM | NM_138766_at | NM_138766 | 1.713 | 440. |
| PAM | NM_138822_at | NM_138822 | 1.712 | 441. |
| PAM | NM_138821_at | NM_138821 | 1.712 | 442. |
| PARP8 | NM_024615_at | NM_024615 | 1.845 | 443. |
| PCOLCE | NM_002593_at | NM_002593 | 1.979 | 444. |
| PCSK1 | NM_000439_at | NM_000439 | 3.694 | 445. |
| PDE4B | NM_001037340_at | NM_001037340 | 3.385 | 446. |
| PDE4B | NM_001037341_at | NM_001037341 | 3.385 | 447. |
| PDE4B | NM_002600_at | NM_002600 | 3.382 | 448. |
| PDE4B | NM_001037339_at | NM_001037339 | 3.381 | 449. |
| PDE6A | NM_000440_at | NM_000440 | 0.397 | 450. |
| PDLIM3 | NM_014476_at | NM_014476 | 1.673 | 451. |
| PDZK1IP1 | NM_005764_at | NM_005764 | 1.938 | 452. |
| PECAM1 | NM_000442_at | NM_000442 | 1.674 | 453. |
| PHLDA1 | NM_007350_at | NM_007350 | 1.807 | 454. |
| PIM2 | NM_006875_at | NM_006875 | 2.422 | 455. |
| PITX2 | NM_153426_at | NM_153426 | 0.177 | 456. |
| PITX2 | NM_000325_at | NM_000325 | 0.177 | 457. |
| PITX2 | NM_153427_at | NM_153427 | 0.177 | 458. |
| PJA1 | NM_001032396_at | NM_001032396 | 1.774 | 459. |
| PJA1 | NM_145119_at | NM_145119 | 1.773 | 460. |
| PJA1 | NM_022368_at | NM_022368 | 1.771 | 461. |
| PLA1A | NM_015900_at | NM_015900 | 2.31 | 462. |
| PLAU | NM_002658_at | NM_002658 | 2.193 | 463. |
| PLEKHC1 | NM_006832_at | NM_006832 | 1.588 | 464. |
| PLEKHG6 | NM_018173_at | NM_018173 | 0.549 | 465. |
| PLEKHO1 | NM_016274_at | NM_016274 | 1.983 | 466. |
| PLIN | NM_002666_at | NM_002666 | 0.59 | 467. |
| PLS3 | NM_005032_at | NM_005032 | 1.544 | 468. |
| PRAP1 | NM_145202_at | NM_145202 | 0.291 | 469. |
| PRDM1 | NM_182907_at | NM_182907 | 1.729 | 470. |
| PRDM1 | NM_001198_at | NM_001198 | 1.728 | 471. |
| PRDX4 | NM_006406_at | NM_006406 | 2.184 | 472. |
| PRKAR2B | NM_002736_at | NM_002736 | 2.26 | 473. |
| PRKD1 | NM_002742_at | NM_002742 | 1.548 | 474. |
| PROCR | NM_006404_at | NM_006404 | 2.195 | 475. |
| PROK2 | NM_021935_at | NM_021935 | 2.72 | 476. |
| PROS1 | NM_000313_at | NM_000313 | 1.69 | 477. |
| PSAT1 | NM_058179_at | NM_058179 | 2.612 | 478. |
| PSAT1 | NM_021154_at | NM_021154 | 2.603 | 479. |
| PSTPIP2 | NM_024430_at | NM_024430 | 2.242 | 480. |
| PTGDR | NM_000953_at | NM_000953 | 0.651 | 481. |
| PTGS1 | NM_080591_at | NM_080591 | 1.854 | 482. |
| PTGS1 | NM_000962_at | NM_000962 | 1.85 | 483. |
| PTGS2 | NM_000963_at | NM_000963 | 2.847 | 484. |
| PTPN13 | NM_080683_at | NM_080683 | 1.748 | 485. |
| PTPN13 | NM_080684_at | NM_080684 | 1.747 | 486. |
| PTPN13 | NM_080685_at | NM_080685 | 1.746 | 487. |
| PTPN13 | NM_006264_at | NM_006264 | 1.731 | 488. |
| PTPRG | NM_002841_at | NM_002841 | 1.743 | 489. |
| RAB23 | NM_016277_at | NM_016277 | 1.615 | 490. |
| RAB31 | NM_006868_at | NM_006868 | 2.108 | 491. |
| RAB34 | NM_031934_at | NM_031934 | 1.693 | 492. |
| RAB38 | NM_022337_at | NM_022337 | 1.725 | 493. |
| RAB3IP | NM_001024647_at | NM_001024647 | 0.627 | 494. |
| RAB3IP | NM_022456_at | NM_022456 | 0.623 | 495. |
| RAB3IP | NM_175623_at | NM_175623 | 0.621 | 496. |
| RAB3IP | NM_175625_at | NM_175625 | 0.587 | 497. |
| RAB3IP | NM_175624_at | NM_175624 | 0.587 | 498. |
| RAI2 | NM_021785_at | NM_021785 | 2.051 | 499. |
| RAPGEFL1 | NM_016339_at | NM_016339 | 0.495 | 500. |
| RARRES1 | NM_002888_at | NM_002888 | 1.782 | 501. |
| RARRES1 | NM_206963_at | NM_206963 | 1.729 | 502. |
| RBKS | NM_022128_at | NM_022128 | 0.547 | 503. |
| RBPMS | NM_001008712_at | NM_001008712 | 1.778 | 504. |
| RBPMS | NM_001008710_at | NM_001008710 | 1.624 | 505. |
| RBPMS | NM_001008711_at | NM_001008711 | 1.624 | 506. |
| RDH5 | NM_002905_at | NM_002905 | 0.614 | 507. |
| RECQL | NM_032941_at | NM_032941 | 1.605 | 508. |
| RECQL | NM_002907_at | NM_002907 | 1.593 | 509. |
| RGL1 | NM_015149_at | NM_015149 | 1.507 | 510. |
| RGS18 | NM_130782_at | NM_130782 | 2.5 | 511. |
| RGS2 | NM_002923_at | NM_002923 | 1.937 | 512. |
| RPA4 | NM_013347_at | NM_013347 | 0.515 | 513. |
| RTN1 | NM_021136_at | NM_021136 | 1.878 | 514. |
| RTN1 | NM_206852_at | NM_206852 | 1.877 | 515. |
| RTN1 | NM_206857_at | NM_206857 | 1.874 | 516. |
| S100A8 | NM_002964_at | NM_002964 | 5.423 | 517. |
| S100P | NM_005980_at | NM_005980 | 2.129 | 518. |
| SAMHD1 | NM_015474_at | NM_015474 | 1.923 | 519. |
| SCG2 | NM_003469_at | NM_003469 | 1.909 | 520. |
| SEC22C | NM_004206_at | NM_004206 | 1.591 | 521. |
| SEC24D | NM_014822_at | NM_014822 | 2.357 | 522. |
| SEMA4D | NM_006378_at | NM_006378 | 1.79 | 523. |
| SERPINA1 | NM_000295_at | NM_000295 | 1.917 | 524. |
| SERPINA1 | NM_001002236_at | NM_001002236 | 1.916 | 525. |
| SERPINA1 | NM_001002235_at | NM_001002235 | 1.916 | 526. |
| SERPINA5 | NM_000624_at | NM_000624 | 0.635 | 527. |
| SERPING1 | NM_000062_at | NM_000062 | 1.991 | 528. |
| SERPING1 | NM_001032295_at | NM_001032295 | 1.99 | 529. |
| SESTD1 | NM_178123_at | NM_178123 | 1.544 | 530. |
| SGK2 | NM_016276_at | NM_016276 | 0.399 | 531. |
| SGK2 | NM_170693_at | NM_170693 | 0.399 | 532. |
| SIGLECP3 | NR_002804_at | NR_002804 | 2.068 | 533. |
| SLAMF1 | NM_003037_at | NM_003037 | 1.827 | 534. |
| SLAMF7 | NM_021181_at | NM_021181 | 2.68 | 535. |
| SLAMF8 | NM_020125_at | NM_020125 | 2.361 | 536. |
| SLC10A2 | NM_000452_at | NM_000452 | 0.484 | 537. |
| SLC16A1 | NM_003051_at | NM_003051 | 0.41 | 538. |
| SLC16A5 | NM_004695_at | NM_004695 | 0.487 | 539. |
| SLC16A9 | NM_194298_at | NM_194298 | 0.253 | 540. |
| SLC20A1 | NM_005415_at | NM_005415 | 0.57 | 541. |
| SLC22A18AS | NM_007105_at | NM_007105 | 0.589 | 542. |
| SLC23A1 | NM_152685_at | NM_152685 | 0.316 | 543. |
| SLC23A1 | NM_005847_at | NM_005847 | 0.315 | 544. |
| SLC23A3 | NM_144712_at | NM_144712 | 0.294 | 545. |
| SLC24A3 | NM_020689_at | NM_020689 | 1.939 | 546. |
| SLC25A34 | NM_207348_at | NM_207348 | 0.498 | 547. |
| SLC31A2 | NM_001860_at | NM_001860 | 1.599 | 548. |
| SLC36A4 | NM_152313_at | NM_152313 | 1.797 | 549. |
| SLC39A5 | NM_173596_at | NM_173596 | 0.628 | 550. |
| SLC6A14 | NM_007231_at | NM_007231 | 8.39 | 551. |
| SLC6A4 | NM_001045_at | NM_001045 | 0.517 | 552. |
| SMOC2 | NM_022138_at | NM_022138 | 1.913 | 553. |
| SOAT1 | NM_003101_at | NM_003101 | 1.848 | 554. |
| SPDYA | NM_182756_at | NM_182756 | 0.547 | 555. |
| SPG20 | NM_015087_at | NM_015087 | 1.655 | 556. |
| SPINK4 | NM_014471_at | NM_014471 | 5.713 | 557. |
| SPIRE2 | NM_032451_at | NM_032451 | 0.565 | 558. |
| ST3GAL5 | NM_003896_at | NM_003896 | 1.815 | 559. |
| ST3GAL5 | NM_001042437_at | NM_001042437 | 1.805 | 560. |
| STCH | NM_006948_at | NM_006948 | 2.221 | 561. |
| SULF1 | NM_015170_at | NM_015170 | 1.865 | 562. |
| SULT1A2 | NM_001054_at | NM_001054 | 0.505 | 563. |
| SULT1A2 | NM_177528_at | NM_177528 | 0.505 | 564. |
| SULT1A3 | NM_177552_at | NM_177552 | 0.639 | 565. |
| SULT1A3 | NM_001017387_at | NM_001017387 | 0.638 | 566. |
| SULT1A4 | NM_001017390_at | NM_001017390 | 0.639 | 567. |
| SULT1A4 | NM_001017391_at | NM_001017391 | 0.638 | 568. |
| TBXAS1 | NM_001061_at | NM_001061 | 2.007 | 569. |
| TBXAS1 | NM_030984_at | NM_030984 | 1.995 | 570. |
| TDO2 | NM_005651_at | NM_005651 | 3.616 | 571. |
| TFPI2 | NM_006528_at | NM_006528 | 3.371 | 572. |
| TGFBI | NM_000358_at | NM_000358 | 2.092 | 573. |
| TICAM2 | NM_021649_at | NM_021649 | 1.616 | 574. |
| TIMP1 | NM_003254_at | NM_003254 | 2.893 | 575. |
| TINAG | NM_014464_at | NM_014464 | 0.498 | 576. |
| TLR1 | NM_003263_at | NM_003263 | 2.816 | 577. |
| TLR2 | NM_003264_at | NM_003264 | 2.436 | 578. |
| TLR7 | NM_016562_at | NM_016562 | 1.92 | 579. |
| TLR8 | NM_138636_at | NM_138636 | 2.912 | 580. |
| TM4SF20 | NM_024795_at | NM_024795 | 0.395 | 581. |
| TMCO3 | NM_017905_at | NM_017905 | 1.54 | 582. |
| TMED6 | NM_144676_at | NM_144676 | 0.286 | 583. |
| TMEM158 | NM_015444_at | NM_015444 | 2.778 | 584. |
| TMEM16F | NM_001025356_at | NM_001025356 | 1.69 | 585. |
| TMEM16J | NM_001012302_at | NM_001012302 | 0.535 | 586. |
| TMEM23 | NM_147156_at | NM_147156 | 1.924 | 587. |
| TMEM45A | NM_018004_at | NM_018004 | 2.496 | 588. |
| TNC | NM_002160_at | NM_002160 | 2.29 | 589. |
| TNFRSF17 | NM_001192_at | NM_001192 | 3.377 | 590. |
| TNFSF13B | NM_006573_at | NM_006573 | 2.249 | 591. |
| TNIP3 | NM_024873_at | NM_024873 | 1.655 | 592. |
| TNNC2 | NM_003279_at | NM_003279 | 0.638 | 593. |
| TOB2 | NM_016272_at | NM_016272 | 0.631 | 594. |
| TPST1 | NM_003596_at | NM_003596 | 1.508 | 595. |
| TPST2 | NM_003595_at | NM_003595 | 1.754 | 596. |
| TPST2 | NM_001008566_at | NM_001008566 | 1.752 | 597. |
| TRIM22 | NM_006074_at | NM_006074 | 2.031 | 598. |
| TRIM9 | NM_015163_at | NM_015163 | 0.615 | 599. |
| TRPM4 | NM_017636_at | NM_017636 | 0.595 | 600. |
| TRPV1 | NM_080705_at | NM_080705 | 0.521 | 601. |
| TRPV1 | NM_080706_at | NM_080706 | 0.519 | 602. |
| TRPV1 | NM_018727_at | NM_018727 | 0.516 | 603. |
| TSEN2 | NM_025265_at | NM_025265 | 0.581 | 604. |
| TUBB6 | NM_032525_at | NM_032525 | 1.777 | 605. |
| TUSC3 | NM_178234_at | NM_178234 | 2.835 | 606. |
| TUSC3 | NM_006765_at | NM_006765 | 2.831 | 607. |
| TWIST2 | NM_057179_at | NM_057179 | 1.572 | 608. |
| TWSG1 | NM_020648_at | NM_020648 | 1.94 | 609. |
| TXNDC5 | NM_030810_at | NM_030810 | 2.318 | 610. |
| TXNDC5 | NM_022085_at | NM_022085 | 2.318 | 611. |
| TYROBP | NM_198125_at | NM_198125 | 2.279 | 612. |
| TYROBP | NM_003332_at | NM_003332 | 2.279 | 613. |
| UCP2 | NM_003355_at | NM_003355 | 1.921 | 614. |
| VAV1 | NM_005428_at | NM_005428 | 1.619 | 615. |
| VEGFC | NM_005429_at | NM_005429 | 1.872 | 616. |
| VNN1 | NM_004666_at | NM_004666 | 2.398 | 617. |
| WARS | NM_173701_at | NM_173701 | 2.382 | 618. |
| WARS | NM_004184_at | NM_004184 | 2.38 | 619. |
| WARS | NM_213645_at | NM_213645 | 2.379 | 620. |
| WARS | NM_213646_at | NM_213646 | 2.378 | 621. |
| WDR41 | NM_018268_at | NM_018268 | 1.774 | 622. |
| WDR78 | NM_024763_at | NM_024763 | 0.55 | 623. |
| WNT5A | NM_003392_at | NM_003392 | 2.709 | 624. |
| XBP1 | NM_005080_at | NM_005080 | 1.899 | 625. |
| XKR4 | NM_052898_at | NM_052898 | 0.543 | 626. |
| YBX2 | NM_015982_at | NM_015982 | 0.518 | 627. |
| ZC3H12A | NM_025079_at | NM_025079 | 1.905 | 628. |
| ZFPM2 | NM_012082_at | NM_012082 | 1.795 | 629. |
| ZNF137 | NM_003438_at | NM_003438 | 0.643 | 630. |
| ZNF575 | NM_174945_at | NM_174945 | 0.554 | 631. |
| ZNF789 | NM_213603_at | NM_213603 | 0.57 | 632. |
| | XM_940819_at | XM_940819 | 6.106 | 633. |
| | XM_940060_at | XM_940060 | 4.181 | 634. |
| | NM_001010919_at | NM_001010919 | 3.954 | 635. |
| | XM_372952_at | XM_372952 | 3.389 | 636. |
| | NM_001008397_at | NM_001008397 | 3.388 | 637. |
| | XM_930497_at | XM_930497 | 3.262 | 638. |
| | XM_938704_at | XM_938704 | 3.26 | 639. |
| | NM_001013618_at | NM_001013618 | 3.215 | 640. |
| | NM_001040077_at | NM_001040077 | 2.978 | 641. |
| | XM_939071_at | XM_939071 | 2.892 | 642. |
| | XM_943820_at | XM_943820 | 2.705 | 643. |
| | XM_943825_at | XM_943825 | 2.705 | 644. |
| | XM_943822_at | XM_943822 | 2.703 | 645. |
| | XM_935086_at | XM_935086 | 2.695 | 646. |
| | XM_935084_at | XM_935084 | 2.694 | 647. |
| | XM_935088_at | XM_935088 | 2.694 | 648. |
| | XM_930293_at | XM_930293 | 2.63 | 649. |
| | XM_936733_at | XM_936733 | 2.619 | 650. |
| | NM_020962_at | NM_020962 | 2.604 | 651. |
| | NM_201613_at | NM_201613 | 2.433 | 652. |
| | NM_201612_at | NM_201612 | 2.42 | 653. |
| | NM_016459_at | NM_016459 | 2.341 | 654. |
| | XM_926979_at | XM_926979 | 2.263 | 655. |
| | NM_015892_at | NM_015892 | 2.183 | 656. |
| | NM_018370_at | NM_018370 | 2.165 | 657. |
| | XM_942376_at | XM_942376 | 2.157 | 658. |
| | NM_080430_at | NM_080430 | 2.034 | 659. |
| | NM_001005410_at | NM_001005410 | 1.927 | 660. |
| | NM_052864_at | NM_052864 | 1.918 | 661. |
| | XM_941100_at | XM_941100 | 1.877 | 662. |
| | XM_932993_at | XM_932993 | 1.846 | 663. |
| | XM_943640_at | XM_943640 | 1.846 | 664. |
| | XM_940833_at | XM_940833 | 1.842 | 665. |
| | XM_944822_at | XM_944822 | 1.842 | 666. |
| | XR_001419_at | XR_001419 | 1.808 | 667. |
| | XR_000584_at | XR_000584 | 1.785 | 668. |
| | NM_007203_at | NM_007203 | 1.772 | 669. |
| | XM_946340_at | XM_946340 | 1.757 | 670. |
| | XM_946339_at | XM_946339 | 1.757 | 671. |
| | XM_942723_at | XM_942723 | 1.757 | 672. |
| | XM_933016_at | XM_933016 | 1.662 | 673. |
| | NM_001040075_at | NM_001040075 | 1.659 | 674. |
| | XM_945072_at | XM_945072 | 1.657 | 675. |
| | NM_016134_at | NM_016134 | 1.652 | 676. |
| | XM_931920_at | XM_931920 | 1.595 | 677. |
| | XM_943451_at | XM_943451 | 1.592 | 678. |
| | XM_931925_at | XM_931925 | 1.592 | 679. |
| | XM_943452_at | XM_943452 | 1.59 | 680. |
| | XM_943019_at | XM_943019 | 1.542 | 681. |
| | XM_936827_at | XM_936827 | 1.54 | 682. |
| | XM_931200_at | XM_931200 | 1.537 | 683. |
| | XM_931194_at | XM_931194 | 1.536 | 684. |
| | XM_926337_at | XM_926337 | 1.535 | 685. |
| | XM_943257_at | XM_943257 | 0.664 | 686. |
| | XM_943532_at | XM_943532 | 0.659 | 687. |
| | XM_933462_at | XM_933462 | 0.658 | 688. |
| | NM_145262_at | NM_145262 | 0.658 | 689. |
| | XM_926967_at | XM_926967 | 0.655 | 690. |
| | NM_152684_at | NM_152684 | 0.651 | 691. |
| | XM_936750_at | XM_936750 | 0.639 | 692. |
| | NM_207482_at | NM_207482 | 0.637 | 693. |
| | XM_940471_at | XM_940471 | 0.632 | 694. |
| | XM_496724_at | XM_496724 | 0.629 | 695. |
| | XM_926453_at | XM_926453 | 0.629 | 696. |
| | XM_944611_at | XM_944611 | 0.626 | 697. |
| | XM_944609_at | XM_944609 | 0.625 | 698. |
| | XM_944919_at | XM_944919 | 0.622 | 699. |
| | XM_932126_at | XM_932126 | 0.619 | 700. |
| | XM_943877_at | XM_943877 | 0.612 | 701. |
| | XM_931100_at | XM_931100 | 0.612 | 702. |
| | XM_931108_at | XM_931108 | 0.611 | 703. |
| | XM_938808_at | XM_938808 | 0.61 | 704. |
| | XM_926245_at | XM_926245 | 0.609 | 705. |
| | NM_173661_at | NM_173661 | 0.588 | 706. |
| | NM_025149_at | NM_025149 | 0.577 | 707. |
| | NM_153270_at | NM_153270 | 0.567 | 708. |
| | NM_015253_at | NM_015253 | 0.564 | 709. |
| | NM_001001704_at | NM_001001704 | 0.518 | 710. |
| | XM_940000_at | XM_940000 | 0.513 | 711. |
| | XM_939562_at | XM_939562 | 0.513 | 712. |
| | XM_085463_at | XM_085463 | 0.513 | 713. |
| | XM_928138_at | XM_928138 | 0.513 | 714. |
| | NM_001018060_at | NM_001018060 | 0.496 | 715. |
| | NM_001013841_at | NM_001013841 | 0.492 | 716. |
| | NM_017720_at | NM_017720 | 0.492 | 717. |
| | NM_138342_at | NM_138342 | 0.475 | 718. |
| | NM_178859_at | NM_178859 | 0.474 | 719. |
| | NM_144704_at | NM_144704 | 0.467 | 720. |
| | XM_930288_at | XM_930238 | 0.464 | 721. |
| | XM_943650_at | XM_943650 | 0.463 | 722. |
| | XM_943644_at | XM_943644 | 0.463 | 723. |
| | XM_938362_at | XM_938362 | 0.463 | 724. |
| | XM_943655_at | XM_943655 | 0.463 | 725. |
| | XM_934563_at | XM_934563 | 0.463 | 726. |
| | XM_934567_at | XM_934567 | 0.463 | 727. |
| | XM_934562_at | XM_934562 | 0.462 | 728. |
| | XM_943653_at | XM_943653 | 0.46 | 729. |
| | XM_934566_at | XM_934566 | 0.459 | 730. |
| | XM_932654_at | XM_932654 | 0.45 | 731. |
| | XM_932662_at | XM_932662 | 0.45 | 732. |
| | XM_932668_at | XM_932668 | 0.449 | 733. |
| | XM_932703_at | XM_932703 | 0.449 | 734. |
| | XM_932711_at | XM_932711 | 0.449 | 735. |
| | XM_932658_at | XM_932658 | 0.449 | 736. |
| | XM_932681_at | XM_932681 | 0.449 | 737. |
| | XM_928205_at | XM_928205 | 0.449 | 738. |
| | XM_932685_at | XM_932685 | 0.448 | 739. |
| | XM_932696_at | XM_932696 | 0.448 | 740. |
| | XM_932688_at | XM_932688 | 0.448 | 741. |
| | XM_932700_at | XM_932700 | 0.448 | 742. |
| | XM_932691_at | XM_932691 | 0.448 | 743. |
| | XM_932317_at | XM_932317 | 0.446 | 744. |
| | XM_927808_at | XM_927808 | 0.446 | 745. |
| | XM_938923_at | XM_938923 | 0.441 | 746. |
| | XR_000535_at | XR_000535 | 0.44 | 747. |
| | XM_932303_at | XM_932303 | 0.437 | 748. |
| | XM_932195_at | XM_932195 | 0.437 | 749. |
| | XM_941939_at | XM_941939 | 0.437 | 750. |
| | XM_932301_at | XM_932301 | 0.437 | 751. |
| | XM_932286_at | XM_932286 | 0.437 | 752. |
| | XM_927596_at | XM_927596 | 0.437 | 753. |
| | XM_932296_at | XM_932296 | 0.437 | 754. |
| | XM_932282_at | XM_932282 | 0.437 | 755. |
| | XM_932268_at | XM_932268 | 0.437 | 756. |
| | XM_932294_at | XM_932294 | 0.437 | 757. |
| | XM_932329_at | XM_932329 | 0.437 | 758. |
| | XM_932291_at | XM_932291 | 0.436 | 759. |
| | XM_932265_at | XM_932265 | 0.436 | 760. |
| | XM_932324_at | XM_932324 | 0.436 | 761. |
| | XM_932280_at | XM_932280 | 0.436 | 762. |
| | XM_932311_at | XM_932311 | 0.436 | 763. |
| | XM_932335_at | XM_932335 | 0.435 | 764. |
| | NR_002815_at | NR_002815 | 0.435 | 765. |
| | XM_936408_at | XM_936408 | 0.431 | 766. |
| | XM_925981_at | XM_925981 | 0.431 | 767. |
| | XM_926814_at | XM_926814 | 0.43 | 768. |
| | XM_932563_at | XM_932563 | 0.415 | 769. |
| | XM_946181_at | XM_946181 | 0.415 | 770. |
| | XM_928053_at | XM_928053 | 0.415 | 771. |
| | XM_942645_at | XM_942645 | 0.415 | 772. |
| | NM_022097_at | NM_022097 | 0.411 | 773. |
| | NM_001013714_at | NM_001013714 | 0.397 | 774. |
| | NM_152672_at | NM_152672 | 0.17 | 775. |

### EXAMPLES

### Example I

A biological sample is obtained via standard biopsy techniques from the ascending colon of a patient diagnosed with Crohn's Disease. A control biopsy is obtained from a matched segment of the colon from a normal subject (not diagnosed with an IBD). The biopsy is obtained at the time of diagnosis. The biological sample is placed in RNAlater™ and stored on ice until processing. Total RNA is prepared utilizing the Qiagen RNeasy mini-column. RNA quality is then assessed using the Agilent 2100 Bioanalyzer. About 400 to about 500 nanograms of total RNA are used. The RNA is then labeled using the Target Amp 1—Round Aminoallyl—aRNA Amplification Kit available from Epicentre (726 Post Road Madison, Wis. 53713 U.S.A.) to prepare cRNA, following the manufacturer's instructions. The TargetAmpl—Round Aminoallyl-aRNA Amplification Kit (Epicentre) is used to make double-stranded cDNA from total RNA. An in vitro transcription reaction creates cRNA target. Biotin-X-X-NHS (Epicentre) is used to label the aminoallyl-aRNA with biotin following the manufacturer's instructions.

The biotin-labeled cRNA target is then chemically fragmented and hybridized to an Affymetrix Genechip Array, HGU133 Plus Version 2 Affymetrix GeneChip, available from Affymetrix (3420 Central Expressway, Santa Clara, Calif. 95051). A hybridization cocktail is prepared, containing 0.034 ug/uL fragmented cRNA, 50 pM Control Oligonucleotide B2 (Affymetrix), 2OX Eukaryotic Hybridization Controls (1.5 pM bioB, 5 pM bioC, 25 pM bioD, 100 pM cre) (Affymetrix), 0.1 mg/mL Herring Sperm DNA (Promega, 2800 Woods Hollow Road, Madison, Wis. 53711 USA), 0.5 mg/mL Acetylated BSA (Invitrogen), and IX Hybridization Buffer. The hybridization cocktail is heated to 99° C. for 5 minutes, to 45° C. for 5 minutes, and spun at maximum speed in a microcentrifuge for 5 minutes. The probe array is then filled with 200 uL of IX Hybridization Buffer and incubated at 45° C. for 10 minutes in the GeneChip Hybridization Oven 640 (Affymetrix) while rotating at 60 rpm. The IX Hybridization Buffer is removed and the probe array filled with 200 uL of the hybridization cocktail. The probe array is then incubated at 45° C. for 16 hrs in a Hybridization Oven rotating at 60 rpm.

The array is then washed and stained using the Fluidics Station 450 (Affymetrix) and the fluidics protocol EukGE-WS2v4_{—}450 (Affymetrix). The stain used is R-Phycoerythrin Streptavidin, available from Molecular Probes. The antibody used is anti-streptavidin antibody (goat) biotinylated, available from Vector Laboratories.

A labeled sample obtained from a single control is used in each batch of microarray experiments. The gene expression results for the new samples within that batch are normalized to the gene expression results for the common control within that batch to provide normalized results that can then be compared between batches.

The probe arrays are then scanned using the Affymetrix GeneChip Scanner 3000, using the Genechip Operating Software Iv4, available from Affymetrix.

Results are interpreted using GeneSpring 7.3 Software, available from Silicon Genetics. Raw data is filtered on an expression level of 10, and then normalized to a uniform internal control RNA from a single healthy control. Each array is then normalized in the same manner. Global scaling is used to adjust the average intensity or signal value of each probe array to the same Target Intensity value (TGT) of about 1500. The internal control genes, GAPDH and B-actin, are used to check the quality of the RNA. The assay quality is determined by comparing the signals of the 3′ probe set to the 5′ probe set of the internal control genes. Acceptable 3′ to 5′ ratios are between about 1 and about 3.

Prokaryotic Spike controls are used to determine whether the hybridization of target RNA to the array occurred properly. To control for chip to chip variation in expression intensities, a common RNA specimen is used, which is labeled and hybridized together with each new batch of biopsy samples.

### Example II

### Gene Expression Profile Determination Using Multiplex PCR

A biological sample is obtained via standard biopsy techniques from the intestines of a patient diagnosed with an inflammatory bowel disease. A control biopsy is obtained from a matched segment of the colon from a subject diagnosed with an IBD, but known to be a “responder” to first line therapy. The biological sample is placed in RNAlater™ and stored on ice until processing. Total RNA is prepared utilizing the Qiagen RNeasy mini-column. RNA quality is then assessed using the Agilent 2100 Bioanalyzer. About 400 to about 500 nanograms of total RNA are used.

PCR primers corresponding to the genes listed in Table 5 and the housekeeping gene GAPDH are synthesized using techniques known in the art. The PCR primers are radiolabeled and selected such that the primers have a primer length of about 18 to about 24 base pairs, and a GC content of about 35% to about 60%, thus having an annealing temperature of about 55° C. to about 58° C. Longer primers of about 28-30 base pairs may be used at higher annealing temperatures. Melting point and primer-primer interactions may be determined using commercially available software such as Primer Premier, available from Premier Biosoft International, 3786 Corina Way, Palo Alto, Calif. 94303-4504. The PCR reaction mixture includes Ix PCR buffer, 0.4 uM of each primer, 5% DMSO, and 1 unit Taq polymerase (Life Technologies, Gaithersburg, Md., USA) per 24 uL reaction volume. Nucleotides (dNTP) (Pharmacia Biotech, Piscataway, N.J., USA) are stored as a 100 mM stock solution (25 mM each dATP, dCTP, dGTP and dTTP). The standard 10×PCR buffer is made as described (Perkin-Elmer, Norwalk, Conn., USA) and contains 400 mM KCL, 100 mM Tris-HCl, pH 8.3 (at 24° C.) and 14 mM MgCl₂. DMSO, BSA and gycerol may be purchased from Sigma Chemical, St. Louis, Mo., USA. The reaction mixtures are then subjected to the following cycling conditions: a first denatureing step of 94° C. for 4 minutes, a denature step at 94° C. for 30 seconds, an annealing step at 54° C. for 30 s, then an extension step at 65 C for one minute. The samples are subjected to 32 cycles, with a final extension step at 65 C for 3 minutes.

Multiplex PCR products are then separated by size on a standard sequencing gel composed of 5% polyacrylamide, and containing 6M urea and 890 mM Tris-borate and 2 mM EDTA. A radiolabeled DNA ladder is used for size determination of each product. Sample is loaded on the gel and the multiplex reaction mixture is electrophoretically separated by size according to standard conditions, for example, 1.5 hours at 2000V, 50 mA current, 20 W power, gel temperature of 51 C. Gene expression of the genes listed in Table 5 is then determined by computer imaging (using GeneScan™ software) of the resultant bands corresponding to PCR products for each gene of interest, quantifying the intensity of each band, and comparing relative quantities of each band of the patient of interest to gene expression in a control subject (the “responder” patient). Both the experimental sample and the control subject results are normalized to GAPDH expression in each sample.

The expression pattern of the patient sample is then compared to the training set of 20 responders and 20 non-responders, using the k-nearest neighbors algorithm, to predict whether the patient is likely to be a “responder” or “non-responder” patient, as described above.

1.-38. (canceled)

39. A method for classifying a subject having or suspected of having an inflammatory bowel disease as a responder or a non-responder to first line treatment, comprising measuring the gene expression in a biological sample obtained from the subject of one or more genes identified in any of Tables 4-8 to obtain a gene expression profile, and comparing the gene expression profile to that of a suitable control.

40. The method of claim 39, wherein the gene expression is determined by a technique selected from the group consisting of PCR, detection of the gene product, and hybridization to an oligonucleotide selected from the group consisting of DNA, RNA, cDNA, PNA, genomic DNA, and a synthetic oligonucleotide.

41. The method of claim 39, wherein the first line treatment is selected from the group consisting of 5-aminosalicylic acid (5-ASA) drugs, corticosteroids, methotrexate, and infliximab.

42. The method of claim 39, wherein a single gene is selected on the basis of being differentially expressed by at least 0.5 fold, or about 1.0 fold, or about 2 fold, or about 3 or about 4 or greater than about 5 fold as shown in any of Tables 4-8.

43. A method for identifying a responder or a non-responder to first line treatment for an inflammatory bowel disease in a subject having or suspecting of having the disease, comprising:
a) obtaining a biological sample from the subject;
b) isolating mRNA from the biological sample;
c) determining a gene expression profile from the biological sample comprising expression values for one or more genes listed in Tables 4-8; and
d) comparing the gene expression profile of the biological sample with a suitable control wherein a comparison of the gene expression profile and the control permits classification of the subject as a responder or a non-responder to the first line treatment for inflammatory bowel disease.

44. The method of claim 43, wherein the gene expression profile comprises at least 2, 5, 10, 15, 20, 25, 30, 35, 40, 45, 50, 60, 70, 80, 90, 100, 150, 200, 250 or more different polynucleotide probes, each different probe capable of hybridizing to a different gene sequence listed in Tables 4-8.

45. The method of claim 43, wherein the one or more genes are selected on the basis of having a fold-change of greater than about 2 or about 3, or about 4 or about 5 as shown in any of Tables 4, 5, 6, 7, or 8.

46. The method of claim 43, wherein the control is a reference gene expression profile selected from the group consisting of a known responder, a known non-responder, and a known refractory.

47. The method of claim 43, wherein the control is selected from one or more housekeeping genes or other gene determined to distinguishable in expression level compared to the same gene, wherein the gene expression values of the subject gene expression profile is determined relative to the control.

48. The method of claim 43, wherein the inflammatory bowel disease is Crohn's Disease.

49. The method of claim 43, wherein the biological sample is colon tissue.

50. The method of claim 43, wherein the biological sample is obtained at the time of diagnosis of the inflammatory bowel disease.

51. The method of claim 43, wherein the first line therapy is selected from the group consisting of 5-aminosalicylic acid (5-ASA) drugs, corticosteroids, methotrexate, 6-mercaptopurine/azathioprine (6-MP/AZA), and infliximab.

52. A gene expression system for identifying a responder or non-responder to first line treatment for an inflammatory bowel disease in a subject having or suspecting of having the disease, comprising a solid support having one or more oligonucleotides affixed to said solid support wherein the one or more nucleotides further comprises at least one sequence selected from those listed in Tables 4-8.

53. The gene expression system of claim 52, further comprising one or more normalization sequences.

54. The gene expression system of claim 52, wherein the inflammatory bowel disease is Crohn's disease or Ulcerative Colitis.

55. The gene expression system of claim 52, wherein the sequences are selected based on the fold change of gene expression in responders compared to non-responders, wherein the one or more genes selected from Tables 4-8 demonstrate a fold change of greater than about 2 or about 3 or about 4 or about 5 as shown in any of Tables 4-8.

56. The gene expression system of claim 52, wherein the solid support comprises an array selected from the group consisting of a chip array, a plate array, a bead array, a pin array, a membrane array, a solid surface array, a liquid array, an oligonucleotide array, a polynucleotide array, a cDNA array, a microfilter plate, and a membrane or a chip.
